(19) United States
(12) Patent Application Publication
(10) Pub. No.: US 2026/0034297 A1
(43) Pub. Date: Feb. 5, 2026

(54) SYSTEM TO CREATE CELLULAR LYSIS AND DELIVER FLUIDS INTRATUMORALLY

(71) Applicant: Syncromune, Inc., Fort Lauderdale, FL (US)

(72) Inventors: Daniel K. Recinella, Waynesville, NC (US); Eamonn P. Hobbs, Fort Lauderdale, FL (US); Stephen T. Kee, Laghey (IE); Timothy B. Gonzalez, Coral Springs, FL (US); Isaias Mena, Fort Lauderdale, FL (US); Dino Kasvikis, Fort Lauderdale, FL (US); Vince Lambert, Fort Lauderdale, FL (US); Eric Hyman, Fort Lauderdale, FL (US); Steve Lepke, Fort Lauderdale, FL (US); Bartosz Korec, Fort Lauderdale, FL (US)

(21) Appl. No.: 19/353,207

(22) Filed: Oct. 8, 2025

Related U.S. Application Data
(63) Continuation-in-part of application No. 19/171,119, filed on Apr. 4, 2025.
(60) Provisional application No. 63/575,527, filed on Apr. 5, 2024.

(51) Int. Cl. A61M 5/158; A61B 18/02; A61K 9/00; A61K 39/395; A61M 25/00

(52) U.S. Cl. CPC A61M 5/158; A61K 9/0019; A61K 39/39558; A61M 25/007; A61M 25/0097; A61B 2018/0293; A61M 2025/0095; A61M 2205/3606; A61M 2205/586

(57) ABSTRACT

The present disclosure provides a system that incorporates a design that has an energy source or sink and a probe for creating cellular lysis and in integral infusion sheath for directing infusion of fluids into the same target where the cellular lysis was performed. The system includes an energy probe and an infusion sheath. In a preferred embodiment, the energy probe is combined with a sheath advancement mechanism housed in a handle. The combined system provides accurate drug infusion into an area of cellular lysis while removing physical manipulation required for other systems where the cellular lysis energy source and sink and the infusion sheath are separate.

### CROSS REFERENCE TO RELATED APPLICATIONS

This application is a continuation-in-part of, and claims the benefit of, and priority to, U.S. Provisional Application No. 63/575,527, filed Apr. 5, 2024, and to U.S. Regular patent application Ser. No. 19/171,119 filed Apr. 4, 2025, and to PCT Application PCT/US2025/023313, filed Apr. 4, 2025, the entire contents of each of which are incorporated herein by reference in their entirety.

### TECHNICAL FIELD

Systems, methods, and devices for cellular lysis treatment and targeted drug infusion post cellular lysis treatment in the field of cancer immunotherapy.

### BACKGROUND

Current clinical approaches for cancer immunotherapy rely on the systemic administration of high doses of one or two biological immunomodulatory agents. Major advances in the treatment of cancer have been made in the last decade by the use of monoclonal antibodies (mAbs) that block immune checkpoint inhibitor molecules such as PD-1, PD-L1 and CTLA-4. Additional immunomodulatory agents such as TLR-9 agonists (CpG oligomers) stimulate immune responses. While agonistic anti-CD40 mAbs or anti-OX40 antibodies reduce pro-inflammatory T-cell responses and induce proliferation of memory and effector T lymphocytes, despite the significant benefits obtained by the use of these mAbs and immunomodulatory agents, singly or in various combinations, sometimes even with chemotherapeutic agents, a substantial fraction of patients still do not respond to the therapy or become refractory to it.

Additionally, the use of high systemic doses of these checkpoint inhibitors may be accompanied by high frequency of adverse events, particularly when used in combination with other immunotherapeutic agents. This may limit the number of agents and doses that can be combined to address the multifactorial nature of tumor immune resistance.

The disclosure of Pending Patent Publication Number US20230404642, published on Dec. 21, 2023, is herein incorporated by reference as if fully set forth herein.

### BRIEF SUMMARY

Systems, methods, and devices described herein relate to, a system for collocating an energy producing or energy withdrawing device for cell lysis of tissue in a target site of a subject and an infusion of a fluid containing immunomodulatory agents into said target site. The system includes: (a) an energy probe configured to emit an energy into or withdraw an energy from the tissue of the target site thereby causing cell lysis of said tissue, and (b) an infusion sheath having a lumen configured to at least partially surround the energy probe, where the infusion sheath is configured to provide a pathway for fluid to travel distally within the lumen and exit a distal portion of the infusion sheath, where the probe is coaxial with or adjacent the infusion sheath and shares a longitudinal axis thereby allowing co-location of the energy probe and the infusion of the fluid from the infusion sheath in the target site. In some embodiments, the infusion sheath includes at least one port in the distal portion of the infusion sheath that is in fluid communication with the pathway of the infusion sheath. In some embodiments, the at least one port is a slit. In some embodiments, the energy probe and the infusion sheath are translatable relative to one another along the longitudinal axis. In some embodiments, where the probe is coaxial with the infusion sheath, the pathway and port are configured to deliver the fluid while the probe is within the pathway at the distal end of the infusion sheath. In some embodiments, the system includes a valve configured to eliminate proximal to distal flow of the fluid during and/or following delivery thereof to the target site. In some embodiments, the system includes a tube in fluid communication with a proximal end of the infusion sheath in fluid communication with the pathway of the infusion sheath at a distal end of the tube, and in fluid communication with a source of the fluid at a proximal end of the tube. In some embodiments, the fluid is a therapeutic agent effective in treating the tissue at the tissue site. In some embodiments, the withdrawal of energy induces cryolysis of tissue at the target site. In some embodiments, the provision or energy induces cryolysis of tissue at the target site. Throughout this disclosure, it should be understood that the energy probe may be one which provides or withdraws energy from target tissue which it is place in contact with. In some embodiments, the system includes a location indicator configured to provide a visual, tactile, or audible indication of the probe's position relative to the infusion sheath's position or vice versa. In some embodiments, the system includes a location indicator configured to provide a visual, tactile, or audible indication of a position of a port of the infusion sheath relative to the probe's position, or vice versa. In some embodiments, the system includes a channel disposed in the exterior surface of the energy probe, the channel configured to slidably couple with a protrusion extending from the interior wall of the infusion sheath, thereby preventing circumferential rotation of the infusion sheath relative to the energy probe. In some embodiments, the system includes a channel disposed at least partially through a wall of the infusion sheath, the channel configured to slidably couple with a protrusion extending from the exterior surface of the energy probe, thereby preventing circumferential rotation of the infusion sheath relative to the energy probe. In some embodiments, the system includes a valve configured to connect an infusion tube with a proximal end of the infusion sheath, so as to provide the infusion of the fluid to the enter lumen of the infusion sheath and/or a plurality of fluid channels disposed within the walls of the sheath. In some embodiments, the infusion sheath has a first inner diameter located at a proximal end of the infusion sheath and a second inner diameter located at a distal end of the infusion sheath. In some embodiments, the system includes an indicator disposed at least partially on or about the energy probe. In some embodiments, the indicator is an audio indicator configured to notify a user that the distal end of the infusion sheath has been advanced to a location relative to the probe that co-locates the fluid delivery with the energy delivered or withdrawn by the probe. In some embodiments, the indicator marks a target location on a proximal end of the system to slidably position the infusion sheath relative to the energy probe to deliver the fluid in a location that is co-located with the energy delivery location of the probe. In some embodiments, the infusion sheath is slidable relative to the longitudinal axis of the energy probe. In some embodiments, the energy probe is slidable relative to the longitudinal axis of the infusion sheath. In some embodiments, the energy probe is a cryoprobe. In some embodiments, the infusion sheath includes a plurality of fluid pathways disposed within a wall of the infusion sheath, where the fluid pathways are configured to release the infusion of the therapeutic agent to flow from a first location of the infusion sheath through a second location of the infusion sheath spaced apart from and located distal to the first location. In some embodiments, the second location is flush with the distal end of the infusion sheath. In some embodiments, the at least one port is an annular orifice. In some embodiments, the infusion sheath includes a polymer. In some embodiments, the infusion sheath includes a metal. In some embodiments, the distance the infusion sheath is slidably moved over the energy probe or the distance the energy probe is moved while at least partially within the infusion sheath is controlled by moving a guide within a slot to a target distance, where the guide extends from the energy probe or the infusion sheath and the slot is at least partially disposed in or extending from the corresponding surface of the energy probe or the infusion sheath. In some embodiments, the infusion sheath has a first inner diameter located at a proximal end of the infusion sheath and a second inner diameter located at a distal end of the infusion sheath. In some embodiments, the second inner diameter is smaller than the first inner diameter, such that said second diameter constricts the flow of fluid by contact with co-axial cryoprobe. In some embodiments, the infusion sheath includes a plurality of fluid pathways disposed through a wall of the infusion sheath, where the fluid pathways are in fluid communication with a source of the fluid at the proximal end of the system, and each pathway is in fluid communication with an associated opening at the distal end of the infusion sheath. Other technical features may be readily apparent to one skilled in the art from the following figures, descriptions, and claims.

Systems, methods, and devices described herein relate to co-locating energy delivery or withdrawal and fluid delivery to tissue at a target site in a patient. In a method according to the disclosure, the method includes: disposing a cryolysis system which includes an energy probe coaxial with and positioned within a lumen of an infusion sheath at least partially into a patient such that a distal end of the energy probe is disposed in the target site, providing energy to or withdrawing energy from tissue thereby lysing at least a portion of a plurality of cells in the target site, slidably moving the infusion sheath in a distal direction along a longitudinal axis of the energy probe from a first position to a second position, where the second position is spaced apart from the first position, delivering an infusion of a fluid to the target site through the infusion sheath, and removing the infusion sheath and the energy probe from the target site. The method in some embodiments includes delivering the fluid out one or more openings in the distal end of the infusion sheath, where such openings are disposed radially through a wall of the infusion sheath to be in fluid communication with a fluid pathway within a lumen of the infusion sheath that extends at a proximal end of the cryolysis system to a fluid source. The method in some embodiments, where delivering the fluid and slidably moving the infusion sheath are done concurrently. The method in some embodiments, where the infusion sheath is in the third position when disposed at least partially into the patient. The method in some embodiments, where a distal end of the infusion sheath is located distal to a distal end of the energy probe when in the third position. The method in some embodiments, where a distal end of the infusion sheath is located proximal to a distal end of the energy probe when in the third position. The method in some embodiments, where a distal end of the infusion sheath is located at a location distal to the distal end of the energy probe when in the second position. The method in some embodiments, where a distal end of the infusion sheath is located at a location proximal to the distal end of the energy probe when in the second position. The method in some embodiments, includes sliding a protrusion extending from the exterior wall of the energy probe within a channel disposed in the infusion sheath to slidably move the infusion sheath from the first position to the second position, where the channel is configured to slidably couple with the protrusion, and optionally, where the protrusion coupled with the channel is configured to prevent circumferential rotation of the infusion sheath relative to the energy probe. The method in some embodiments, includes sliding a protrusion extending from the exterior surface of the energy probe within a channel disposed at least partially through a wall of the infusion sheath to slidably move the infusion sheath from the first position to the second position, where the channel is configured to slidably couple with the protrusion, and optionally, where the protrusion coupled with the channel is configured to prevent circumferential rotation of the infusion sheath relative to the energy probe. The method in some embodiments includes determining the second position by aligning a portion of the proximal end of the sheath relative to a portion of a proximal end of probe according to an audio, tactile, or visual indicator. The method in some embodiments, where delivering the infusion of a therapeutic agent to the target tissue occurs through a valve configured to connect an infusion tube with a proximal end of the infusion sheath, so as to provide fluid through a pathway within the infusion sheath and out the one or more openings of the infusion sheath. The method in some embodiments, where the energy probe is a cryoprobe and withdrawing energy from the tissue at the tissue site at least partially freezes the tissue thereby lysing at least a portion of the plurality of cells in the target site. The method in some embodiments, where delivering the infusion of a fluid to the target tissue includes opening a valve at the proximal end of the system in the fluid pathway of the fluid that connects an infusion fluid source with a proximal end of the infusion sheath. The method in some embodiments, where the infusion sheath is configured to form a seal with the energy probe when in the first position such that the infusion of therapeutic agent is retained in the infusion sheath, and where the infusion sheath is configured to deliver the infusion of therapeutic agent to the target tissue when in the second position. The method in some embodiments, where a distal end of the infusion sheath is located proximal to the distal end of the energy probe when in the first position. Other technical features may be readily apparent to one skilled in the art from the following figures, descriptions, and claims. In some embodiments, the therapeutic agent is a cancer therapeutic agent. In some embodiments, the first inner diameter is larger than the second inner diameter, and where the first inner diameter tapers to the second inner diameter in a location between a proximal end of the infusion sheath and the distal end of the infusion sheath. In some embodiments, the second inner diameter forms a fluid tight seal with the outer diameter of the energy probe, such that when a portion of the energy probe is in a first position disposed within the first region of the infusion sheath having the second inner diameter, the fluid does not flow from the inner lumen of the infusion sheath through the distal end of the infusion sheath, and where, when the energy probe is in a second position at least partially disposed the second region of the infusion sheath having the first inner diameter and not in the first region of the infusion sheath the infusion sheath having the second inner diameter, the fluid in the inner lumen of the infusion sheath is in fluid communication with the target site. In some embodiments, the indicator is a visual distance and/or rotational indicator located on the system that indicates to a user that a distal end of the infusion sheath has been advanced to a location relative to the probe that co-locates the fluid delivery with the energy delivered or withdrawn by the probe. In some embodiments, the first inner diameter is larger than the second inner diameter, and where the first inner diameter tapers to the second inner diameter in a location between the proximal end of the infusion sheath and the distal end of the infusion sheath. In some embodiments, the second inner diameter is substantially the same as the outer diameter of the energy probe, such that when a portion of the energy probe is disposed within the second inner diameter, fluid does not flow from the inner lumen of the infusion sheath through the distal end of the infusion sheath, and where the first inner diameter is larger than the outer diameter of the energy probe, such that when the energy probe is at least partially disposed only in the portion of the energy probe having the first inner diameter, the inner lumen of the infusion sheath is in fluid communication with the target site. In some embodiments, the opening is selected from a group consisting of a slit and an annular orifice. The method in some embodiments, where the one or more openings is a slit. The method in some embodiments includes slidably moving the infusion sheath in a proximal direction along the longitudinal axis of the energy probe from the second toward the first position, where delivering the fluid and slidably moving in this proximal direction are done concurrently. The method in some embodiments, includes slidably moving the infusion sheath distal to the second position to a third position, where when in such third position buckling of the infusion sheath is reduced relative to when in the first or second positions and/or mechanical strength of the system at the location of the energy probe is enhanced relative to the energy probe alone without the sheath distal advancement, where the enhanced mechanical strength is provided about the distal end of the energy probe upon disposing the cryolysis system in the patient. The method in some embodiments, where the indicator marks a target location to slidably align the infusion sheath and the energy probe thereby co-locating delivery of the fluid with the energy delivered or withdrawn by the probe. In further embodiments according to this disclosure, there is provided a mechanism for advancing and retracting the sheath in relation to the energy probe and targeted treatment site. Other technical features may be readily apparent to one skilled in the art from the following figures, descriptions, and claims.

### BRIEF DESCRIPTION OF THE SEVERAL VIEWS OF THE DRAWINGS

Figures illustrated herein are exemplary block drawings showing relative positions of certain elements described herein. However, the figures do not show the exact couplings or connections that may or may not exist between elements. Additionally, in some drawings, internal and external features may be shown in the same renderings for conceptualizing the overall concepts and not for conceptualizing them from an engineering perspective.

FIG. 1A illustrates an example of a cryolysis system in a first position comprising a probe and an infusion sheath comprising at least one port, according to embodiments described herein.

FIG. 1B illustrates an example of the cryolysis system shown in FIG. 1A in a second position, according to embodiments described herein.

FIG. 2A illustrates an example of a cryolysis system in a first position comprising a probe and an infusion sheath having a plurality of lumens disposed in the wall of the infusion sheath, according to embodiments described herein.

FIG. 2B illustrates an example of the cryolysis system shown in FIG. 2A in a second position, according to embodiments described herein.

FIG. 3A illustrates an example of a cryolysis system in a first position comprising a probe and an infusion sheath having two or more inner diameters and a slide coupled with a channel, according to embodiments described herein.

FIG. 3B illustrates an example of a cutaway view of the cryolysis system shown in FIG. 3A with the energy probe extended beyond the infusion sheath, according to embodiments described herein.

FIG. 3C illustrates an example of a second cutaway view of the cryolysis system shown in FIG. 3A in a second position, according to embodiments described herein.

FIG. 4A illustrates an example of an energy probe and an infusion sheath system inserted into a target tissue, according to embodiments described herein.

FIG. 4B illustrates an example of the energy probe and infusion sheath of FIG. 4A delivering energy to the target tissue site, according to embodiments described herein.

FIG. 4C illustrates an example of the area of cellular lysis created from the energy that was delivered by the energy probe and infusion sheath of FIG. 4B in the target zone of the target tissue, according to embodiments described herein.

FIG. 4D illustrates an example of the energy probe and infusion sheath of FIG. 4C with the infusion sheath positioned in the same target location that has experienced cellular lysis, and delivering therapeutic agent in the same area according to embodiments described herein.

FIG. 5A illustrates an example of a cryolysis system comprising an energy probe and an infusion sheath in a first position, according to embodiments described herein.

FIG. 5B illustrates a second view of the cryolysis system shown in FIG. 5A in a second position, according to embodiments described herein.

FIGS. 6A and 6B show an Oncolysis Infusion Device (OID) with a first embodiment of a sheath advancement mechanism, for use with an angled energy probe.

FIGS. 7A and 7B show an Oncolysis Infusion Device (OID) with a second embodiment of a sheath advancement mechanism, for use with an angled energy probe.

FIGS. 8A and 8B show an Oncolysis Infusion Device (OID) with a third embodiment of a sheath advancement mechanism, for use with an angled energy probe.

FIGS. 9A and 9B show an Oncolysis Infusion Device (OID) with a fourth embodiment of a sheath advancement mechanism, for use with an angled energy probe.

FIG. 10A-10D show an Oncolysis Infusion Device (OID) with a fifth embodiment of a sheath advancement mechanism.

FIG. 11 is an Oncolysis Infusion Device (OID) with a sixth embodiment of a sheath advancement mechanism for use with a straight, non-angled energy probe.

### DETAILED DESCRIPTION

As used herein, the term “about” means within +10% of the value it modifies. For example, “about 1” means “0.9 to 1.1”, “about 2%” means “1.8% to 2.2% ”, “about 2% to 3%” means “1.8% to 3.3%”, and “about 3% to about 4%” means “2.7% to 4.4%.” Unless otherwise clear from the context, all numerical values provided herein are modified by the term “about”.

As used herein, the term “cellular lysis” refers to the breaking down of the membrane of a cell, through the application of energy to or withdrawal of energy from a targeted area. Cellular lysis may encompass diverse mechanisms of cell death including necrosis, necroptosis, apoptosis, pyroptosis, etc. For example, cellular lysis may cause the contents of the cells to be released into the micro-environment surrounding the lysed area and initiate an immune response by diverse mechanisms.

As used herein, the term “cryolysis” refers to a process that uses localized, extreme cold to partially destroy a target tissue. A target tissue may be an abnormal or diseased tissue, such as a tumor. Cryolysis, may be performed in methods described herein. Cryolysis may be, similar to cryoablation, but cryolysis does not aim to ablate an entire tumor. Accordingly, during administration of a therapy that causes cell lysis by cryolysis, cryoprobes are positioned adjacent to, or within (e.g., intratumoral positioning), a target tissue in such a way that a freezing process will partially destroy abnormal or diseased tissue, such as a tumor. In some embodiments of the present disclosure, a goal of cryolysis is not to achieve full ablation of the tumor tissue (i.e., cryoablation), but to elicit a partial necrotic zone within a tumor to release sufficient amounts of tumor specific antigens (TSA), tumor associated antigens (TAA) and damage associated molecular patterns (DAMPs) to initiate an immune response. Accordingly, cryolysis may differ from cryoablation in methods of administration including, but not limited to, minimum temperature achieved in a target tissue treatment zone, rate of cooling of a target tissue treatment zone (which is related to, among other things, rate of cooling of a cryotherapy device, such as a cryoprobe), diameter of an ice ball formed at a target tissue treatment zone, diameter of an isotherm within an ice ball that reaches a critical temperature for necrosis and apoptosis, duty cycle of a cryotherapy device, speed of thaw, and duration of freeze-thaw cycles

As used herein, the term “cryoprobe” refers to a hollow needle or elongated tube through which a cooled, thermally conductive, fluid is circulated. A thermally conductive fluid may be any acceptable cryogen used in the field, for example, argon, nitrous oxide, carbon dioxide, or liquid nitrogen.

As used herein, the term “energy probe” or “probe” refers to any structure that is designed to be inserted into soft tissue or bone and is used to transmit energy from an energy source to a target site or to withdraw energy from a target site to a site of cooling. For example, the energy probe may facilitate cellular lysis by applying or withdrawing the energy to or from the surrounding tissue. In some embodiments, this energy source is a cryoprobe. In some embodiments, the energy probe is for creating Irreversible Electroporation (IRE) in the target tissue. In some embodiments the energy probe is for delivering Pulsed Electric Fields (PEF) to the target tissue. A cryoprobe is an example of an energy probe. The terms “probe,” and “energy probe” are used interchangeably throughout this disclosure. As used herein the term “probe” is an energy probe. Alternatives and embodiments of energy probes are described further infra.

As used herein, the term “energy source” refers to any system that generates a form of energy that can be transmitted or which provides a locus for transmission of heat from a tissue to a heat sink such as a cryoprobe. For example, the energy source may be selected from a group consisting of radiofrequency, microwave, ultrasound, cryo, electrical, any other energy source or sink known to those skilled in the art, and any combination thereof applied to a structure, such as a probe.

As used herein, the term “infusion sheath” or, synonymously, “infusible sheath,” refers to a channel where the flow of fluid is established. For example, this sheath may comprise different materials such as polymer or metal and can be of varying design such as a single tube, or a multi-lumen tube. A characteristic that defines the infusion sheath is the ability for fluid to flow through an inlet that is then carried distally through an inner portion of the sheath having an egress to thereby permit the flow of fluid out of the sheath and into the target site. Alternatives and embodiments of infusion sheaths are described further infra.

As used herein the terms “drug”, “biologic”, and “therapeutic agent” may be used interchangeably to refer to pharmacologically active molecules that are used to treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds such as immunomodulatory compounds.

As used herein, the term “oncolysis” refers to a modified state of tissue within a tumor where a portion of cells have been lysed and their contents have flowed into the surrounding environment. This occurs while, at the same time, portions of the target area remain intact with pathways, such as lymphatics, still being viable for transporting material from the tumor environment into the lymphatic system where T-cells encounter Antigen Presenting Cells (APC's).

As used herein, the terms “subject” or “patient” may be used interchangeably and refer to any animal, including a mammal, to which a provided method and/or composition is or may be administered, e.g., for experimental, diagnostic, prophylactic, and/or therapeutic purposes.

The term “mammal” as used herein, encompasses any mammal. Examples of mammals include, but are not limited to, cows, horses, sheep, pigs, cats, dogs, mice, rats, rabbits, guinea pigs, monkeys, humans, etc. The subject is preferably a human. In some embodiments a subject is suffering from or is susceptible to one or more disorders or conditions. In some embodiments, a subject displays one or more symptoms of a disorder or condition. In some embodiments, a subject has been diagnosed with one or more disorders or conditions. In some embodiments, a disorder or condition is or includes a proliferative disease such as cancer. In some embodiments, a cancer is a solid tumor cancer. In some embodiments, a subject is receiving or has received certain therapy to diagnose and/or to treat a disease, disorder, or condition.

Traditional cancer immunotherapeutic approaches may provide benefits to a limited fraction of patients. For example, this may be due to systemic delivery of drugs which is limited in effectiveness and is associated with toxicity and adverse events. However, by delivering drugs intratumorally the amount of drug required is reduced compared to systemic delivery. Intratumoral delivery immediately following cellular lysis may carry the intracellular components, i.e., tumor specific antigens (TSA), tumor associated antigens (TAA), damage associated molecular patterns (DAMPs) and other cellular components released by the breakdown of the cellular wall, into the tumor environment and lymphatic system thereby initiating an immune response.

Another approach includes applying or withdrawing energy to or from the tumor locally. In some embodiments, different energy sources and sinks and probes are used to create cellular lysis. In some embodiments, the energy source or sink used to create cellular lysis is a cryoprobe. Cryolysis, as described herein, may refer to a process that uses localized, extreme cold to partially destroy a target tissue, e.g., an abnormal or diseased tissue, such as a tumor. Thus, cryolysis, as performed in methods described herein, may be similar to cryoablation, but cryolysis does not aim to ablate an entire tumor. For example, a goal of cryolysis may not be to achieve full ablation of the tumor tissue (i.e., cryoablation), but to elicit a partial necrotic zone within a tumor to release sufficient amounts of tumor specific antigens (TSA), tumor associated antigens (TAA) and damage associated molecular patterns (DAMPs) to initiate an immune response. In some embodiments, fluid pathways within the tumor environment are maintained and not destroyed as would be the case with cryoablation.

However, traditional approaches of localized treatment of cancer by injecting drugs locally have not been optimized, because they fail to infuse drug in the same location where the energy source was placed. Surprisingly, as described herein, by infusing drug into a cancer target site (i.e., intratumoral administration) immediately post cell lysis and accurately in the center of the treatment zone of the target site may result in optimization of the entrainment of the released antigens and other cellular components. Additionally, intratumoral administration may allow for a significant reduction in dose, which reduces cost and the associated risk of toxicity and occurrence of an adverse event. In some embodiments, the reduced dose of drug is delivered accurately into the target cellular lysis zone in order to effectively “wash” the tumor micro-environment of cellular components that have flowed into the area due to the cellular lysis treatment.

Systems, devices, and methods as described herein relate to the infusion of an immunotherapeutic drug into the same target site location of the cryolysis treatment. In some embodiments, the entrainment of cellular components into the peritumor lymphatic system is optimized via infusion of immunotherapeutic drug into the same target site location of the cryolysis treatment.

A principle of hydrodynamics is that liquids will flow from areas of higher pressure to areas of lower pressure, via the paths of least resistance and highest compliance. In some embodiments, during an infusion the target tissues exhibit resistance and compliance along a varying scale of elasticity. In some embodiments, the paths of least resistance change as the various pathways change in resistance and compliance. In some embodiments, the paths of least resistance become saturated with fluid and exhibit increasing resistance.

Normal, healthy tissue primarily uses the lymphatic drainage system to maintain fluid balance within the tissue. The lymphatic drainage vessels are typically the primary drainage pathway for interstitial and extravasated fluid. When the lymphatic drainage is compromised, edema may occur as a result. Metastatic tumors may extensively utilize the lymphatic system to traffic and message. For example, metastatic tumors are often hyperlymphatic when compared to healthy tissue or to earlier stage tumors. Tumors may be associated with tertiary lymphoid structures (TLS) either intratumorally, peritumorally, or in nearby tissue. TLS may communicate with the tumor and with secondary lymphoid structures such as tumor draining lymph nodes (TDLNs) via lymphatic vessels. The lymphatic tissue drainage system may present a significant path of least resistance for the tumor tissue resolving the interstitial, extravasated fluid overload that is created by the drug infusion into the treatment zone. Fluid flow through tissue medium has been modeled with Darcy's Law. In the context of cancer, Darcy's Law is linked to the interstitial fluid, the interstitial fluid pressure, and the hydraulic conductivity of the tumor environment, despite being originally derived to calculate flow through porous media. Darcy's Law describes that fluid flow will occur from a high-pressure area to a low-pressure area, based on the pressure difference and the hydraulic conductance of the medium. Hydraulic conductance is the capacity of a porous material to allow fluid to flow under the effect of pressure differentials. Higher pressure differentials between fluid inlets and outlets may create higher fluid flowrates.

Intratumoral vasculature may be tortuous, poorly organized, and have inconsistent vessel diameters with more prevalent branching compared to other normal tissue. For example, the vasculature and lymphatics within the center of the tumor are compressed due to the interstitial pressure within the tumor. The compression may create a high-pressure localization within the center of the tumor, thereby driving fluid flow within the tumor to the low-pressure draining system, represented by the peritumoral lymphatic vessels. Following Darcy's Law, the path from the center of the tumor to the peritumoral lymphatic vessels is the path of least resistance for fluid to flow from the tumor.

In some embodiments, an infusion rate between 1-3 ml/min of immunotherapeutic drug to the target site is used. The infusion rate may be selected to optimize for immunotherapeutic drug to first mix with the relatively lower resistance immediately surrounding lysed cell debris in the treatment zone. In some embodiments, the lysed cell debris includes tumor antigens, DAMPS, and other cellular contents. In some embodiments, once the lysed treatment zone has been saturated, the infused immunotherapeutic drug will flow via the paths of least resistance into the surrounding intact tumor tissue. In some embodiments, the infused immunotherapeutic drug also exits via the first available lymphatic vessel drainage pathway and blood vessel pathways. When the compliance limit of the tissue is reached, the continued infusion pressure reaches an equilibrium, where outgoing flow volume out of the tumor via the draining lymphatic vessels and blood vessels is approximately equal to the incoming flow volume being infused into the tumor. In some embodiments, the equilibrium point varies based on the intratumoral and peritumoral tissue compliance. In some embodiments, the equilibrium point will be reached at or before 5 ml of drug has been infused.

In some embodiments, at the equilibrium point, the continued infusion flow will saturate and distend the tumor tissue to a limit of its compliance. In some embodiments, the infused drug and any diffusible entities have mixed with and washed out into the surrounding intratumoral tissue, peritumoral tissue, and/or lymphatic drainage vessels. In some embodiments, the diffusible entities include lysed tumor cell contents, tumor antigens, DAMPS, and any combination thereof. In some embodiments, most of the excess fluid mixture of infused drug and diffusible entities drain out of the treatment zone and reach their cellular targets within the tumor microenvironment, TLSs, and TDLNs via the draining lymphatic vessels. In some embodiments, when the infusion is completed, the pressure head of the infusion is terminated and the distended and elastic intratumoral and peritumoral tissues drain the mixture of excess drug and debris fluid via the path of least resistance and return to their resting, pre-injection pressures. In some embodiments, the path of least resistance is the lymphatic vessel drainage system.

Current systems designed for intratumoral infusion of immunotherapeutic drugs alonc include a multi-side hole needle with a closed distal end that creates jets of infusate. However, these systems are separate from the energy probe and are placed independently. With this type of system, locating the end of the needle in the same location where the energy system was deployed may not be accurate and may allow for errors in placement, making the infusion of liquid miss the intended target. Embodiments of the disclosure described herein directly address this consideration by including a mechanism for precisely advancing and retracting the sheath in relation to the energy probe and target site.

Another current method of injecting immunostimulatory agents intratumorally includes placing a needle into the tumor and injecting without delivering or withdrawing energy prior to injections. With this method, accuracy of placement for infusion is not considered and the resulting drug infusion is inefficient.

A method of creating lysis by using temperatures around −40° C. and then delivering an effective amount of selected antigen presenting cells intratumorally or in proximity to the tumor or cancerous tissue when the bioavailability of the cancer-specific antigens in the bloodstream is at the maximum value has also been explored. In this method, the infusion may not be done within the same target area where the energy is delivered or withdrawn and not performed immediately post energy transfer. Therefore, the accuracy of infusion may not be considered and may have a deleterious effect on the outcome of the treatment.

Current methods of cellular ablation such as irreversible electroporation (IRE) and pulsed electric fields (PEF) aim to cause cellular destruction by the use of electric fields. This modality requires that electrical pulses travel from a probe to either another probe or a grounding pad that is placed in another area on the patient. The exact area of cellular destruction is not always apparent. Post treatment drug infusion is achieved by placing a needle into an area near the treatment zone. In some embodiments, the needle is not part of the system and requires user skill to place it accurately. This method may be inaccurate for drug delivery into the area of cellular lysis and may result in less effective immunogenic results. Embodiments of the disclosure described herein directly address this consideration by including a mechanism for precisely advancing and retracting the sheath in relation to the energy probe and target site.

Other methods cryoablate by placing the cryoprobe into the target tissue and causing cellular destruction by freezing the tissue with long freezing cycles lasting 8 to 10 minutes. These freezing cycles are often repeated numerous times to destroy the entirety of the tumor. A needle is then placed approximately where the ablation was performed, without accuracy and without assured co-location with the cryoprobe. It is usual that the needle is not part of the system and requires user skill to place it accurately. This method may be inaccurate and may not provide the expected immunogenic response. Further, long and repeated freeze/thaw cycles are used in cryoablation where the entirety of the tumor is destroyed. Lymphatic pathways would be destroyed as well.

In contrast to the methods noted in the prior several (5) paragraphs, the system of the present disclosure comprises a design that includes an energy probe combined and integral with an infusion sheath for infusing agents accurately into selected target. The energy probe and the agents infused, thus, are co-located in a selected target. In some embodiments, the configuration of the infusion sheath is not dependent on the drugs to be infused. For example, the drugs infused may be of any single drug or multiple mixture of drugs as the user deems to be appropriate for the subject at that time. In other words, the infusion sheath may be agnostic to the drug or mixture of drugs passed through the sheath. In some embodiments, the infusion sheath is configured to permit a specific type of drug to pass through the sheath. For example, in a drug formulation having a higher viscosity, the infusion sheath may be formulated with a larger diameter thereby reducing the pressure needed to infuse the drug formulation into a user.

### Probe

Numerous cryosurgical instruments including cryoprobes, cryosurgical probes, cryosurgical ablation devices, cryostats and cryocoolers have been used for cryosurgery. These devices typically use the principle of Joule-Thomson expansion to generate cooling, taking advantage of the fact that cryogenic fluids, when rapidly expanded, become extremely cold. In some embodiments, a high-pressure gas mixture is expanded through a nozzle inside a small cylindrical shaft or tube typically made of steel. The Joule-Thomson expansion cools the steel tube to a cold temperature very rapidly. In some embodiments, the cooled cryosurgical probes are brought into contact with diseased tissue, forming ice balls and freezing the diseased tissue. Properly performed cryosurgical procedures allow cryoablation of the diseased tissue without undue destruction of surrounding healthy tissue.

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryosurgical treatment can be used to treat both benign and cancerous tissues, e.g., prostate cancer and benign prostate disease. Cryosurgery also has gynecological applications in treatment of the uterus or fibroids. In addition, cryosurgery may be used for the treatment of a number of other diseases and conditions including, but certainly not limited to, breast cancer, liver cancer, renal cancer, gliobastomas such as glioblastoma multiforme (GBM), glaucoma and other eye diseases.

In some embodiments, a cryolysis therapy is mediated by an intratumoral cryolysis device comprising a cryoprobe type of probe for contacting and freezing tumor cells during freeze-thaw cycles. In some embodiments, a cryogen circulates within a cryoprobe. In some embodiments, a cryogen is selected from the group consisting of: argon, nitrous oxide, carbon dioxide, and liquid nitrogen. In some embodiments, administration of a cryolysis therapy comprises at least one, at least two, or at least three cycles of freeze-thaw. In some embodiments, administration of a cryolysis therapy comprises one cycle of freeze-thaw. In some embodiments, administration of a cryolysis therapy comprises two cycles of freeze-thaw. In some embodiments, administration of a cryolysis therapy comprises three cycles of freeze-thaw.

In some embodiments, the probe is an energy probe. In some embodiments, the energy source or sink used to create cellular lysis is selected from a group consisting of ultrasound, cryolysis, radiation, radiofrequency, high intensity focused ultrasound (HIFU), microwave, pulsed electric fields, and any combination thereof. Other energy sources known to those of skill in the art may also be used to emit energy at a target site within a patient. In some embodiments, the energy probe is a cryoprobe source configured to generate cryolysis therapy by the flow of a cryogenic fluid through the probe that mediates tumor cellular lysis by cryolysis.

In some embodiments, the cryoprobe is configured to be any diameter that is commonly used in the field for intratumoral cryotherapy. In some embodiments, a cryoprobe has a diameter of about 1.5 mm to about 3.4 mm. In some embodiments, a cryoprobe has a diameter of about 1.5 mm. In some embodiments, a cryoprobe has a diameter of about 3.4 mm. In some embodiments, a cryoprobe diameter is selected to enable a rapid cooling rate. In some embodiments, achieving a rapid cooling rate requires that a cooling probe achieves a temperature of about −40° C. or colder, about −50° C. or colder, or about −60° C. or colder, in less than a minute. In some embodiments, a cryoprobe surface may reach −130° C. to −150° C. and as low as −190° C. as needed for rapid cooling of target tissue. In some embodiments, cryoprobe diameter is selected to enable efficient cryolysis of a target tissue such that the cryoprobe effectively elicits a partial necrotic zone within a target tissue, which can be a tumor, to release sufficient amounts of TSA and DAMPs to initiate an immune response. In some embodiments, the probe is coaxial with the infusion sheath. The probe sizes may range from 1 mm in outer diameter to 3 mm in outer diameter.

In a non-limiting example, the energy being delivered or withdrawn through the probe may create cellular lysis by necrosis, thereby disrupting the cellular walls and facilitating the release of antigens and other cellular components into the tumor micro-environment. In some embodiments, the drug infusion sheath facilitates the accurate infusion of drugs into the same location of the target site where the cellular lysis was created. In some embodiments, the probe passes through the conduit for drug infusion such that the probe and the drug infusion sheath coincide with each other. Embodiments of the disclosure described herein directly address this consideration by including a mechanism for precisely advancing and retracting the sheath in relation to the energy probe and target site. Thus, the location the drug infusion coincides with the cellular lysis treatment area and errors in directing the drug infusion into the target treatment zone are reduced or eliminated.

In some embodiments, the energy probe is disposed within a handle adapted to be handheld or fixed to a mechanical fixture which helps steady the energy probe as it is guided to the target zone in a subject. The mechanical fixture may be a securely fixed pole, or support on a pole, or a more sophisticated support and guidance system.

In some embodiments, there is provided a cryoprobe sheath in movable concentric axial alignment with and covering at least a portion of a cryoprobe when inserted into an end of the sheath proximal the handle, such that advancement or retraction of the sheath in relation to a distal end of the cryoprobe causes the distal end of the cryoprobe to emerge from or be covered by a distal end of said sheath.

In some embodiments, there is provided a sheath advancement and retraction mechanism which permits precise mechanical advancement, retraction, or both, of the sheath with respect to a distal end of the cryoprobe.

In some embodiments, a conduit from a source of fluid is in sealed fluid communication with the sheath such that when a distal end of the sheath is not advanced beyond a set point in relation to the cryoprobe, fluid from the fluid source is restricted from flowing through the sheath, and when the distal end of the sheath is advanced to a set point in relation to the cryoprobe, fluid from the fluid source is permitted to flow from the fluid source, through and out of the distal end of the sheath.

In some embodiments, a source of compressed cryogenic gas which endothermically expands within the cryoprobe to cause fracture of tissue placed in contact with said cryprobe.

### Infusion Sheath

In some embodiments, the energy probe is surrounded by an infusion sheath, and is thus coaxial with said energy probe for infusion of a fluid (i.e. the drug) into the target site, which can include target tissue or be at the treated area in instances where the probe is activated prior to the fluid infusion to the same co-located site. In some embodiments, the infusion sheath comprises a lumen coaxial with the probe which resides in such lumen. In use, in some embodiments, the probe translates distally out of the sheath into a target site (e.g. a tumor), where a cryo energy sink or other energy from the probe is withdrawn from or delivered to the tissue of the target site. The infusion sheath can translate distally over the probe and deliver a treatment fluid out of openings (e.g. slits or holes or elongated holes) in the distal end or in a distal portion of the infusion sheath to the target site while keeping the probe substantially stationary to co-locate the fluid with the location of energy (e.g. cryo) delivery by the probe. The delivery of the treatment fluid may occur while the infusion sheath is moving distally and/or proximally relative to the probe, or at a point or at several intermediate points when the infusion sheath is stationary relative to the probe, at the discretion of the user to co-locate the fluid relative to the probe's energy delivery or withdrawal.

In some embodiments, the outer diameter of the probe may be smaller than the inner diameter of the sheath, creating a space for the fluid to travel distally to exit one or more openings or ports at the distal end of the infusion sheath, which may include one or more slits or one or more holes or one or more elongated holes at a distal portion of the infusion sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location. In some embodiments, the relative location of the infusion sheath and probe is fixed by mechanical means described herein to either permit or prevent infusion flow at the target site.

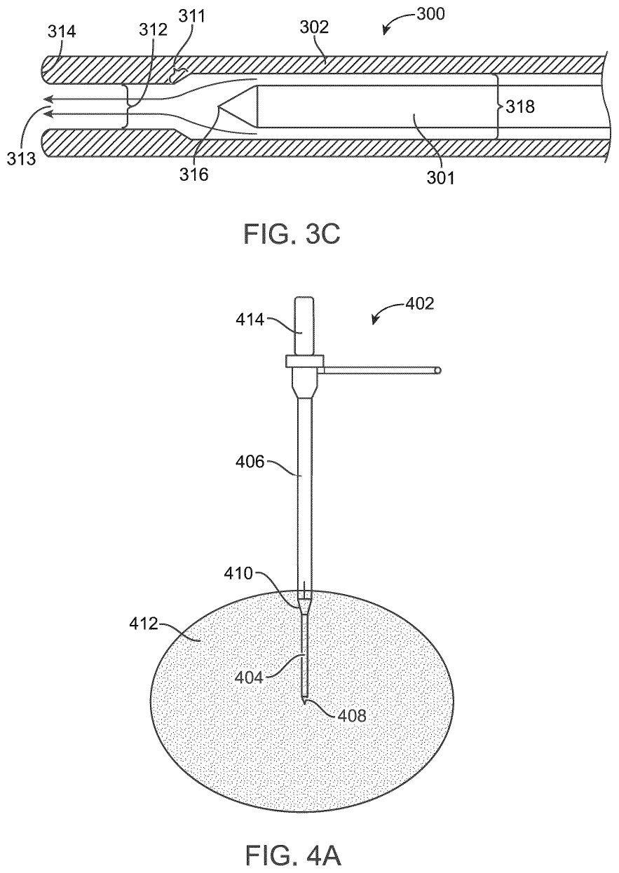

In some embodiments, the outer diameter of the probe is tapered to increase from the distal tip in a proximal direction. For example, the distal tip may have a first outer diameter that is a smaller outer diameter than the diameter located proximal thereto. In such embodiments, the infusion sheath may have a constant inner diameter at the distal end thereof such that the distal translation of the infusion sheath over the distal tip of the probe which creates an open pathway to allow the fluid to exit the opening of the system between the infusion sheath and the probe. In some embodiments, a distal portion of the probe is a constant outer diameter, and the infusion sheath comprises a distal portion having a smaller inner diameter than a second distal portion proximal thereto, for example as described and shown in FIGS. 3A-3C.

In some embodiments, the difference in the outer diameter of the probe and the inner diameter of the sheath creates sufficient opening for the fluid to travel to the distal portion of the sheath. In some embodiments, the fluid travels distally to exit the distal tip of the sheath. In some embodiments, the one or more openings may be included in the sheath that are or extend proximal to the distal tip of the sheath. In some embodiments, the sheath is substantially tapered at the distal end such that the inner diameter of the sheath is substantially the same as the outer diameter of the probe (although still translatable relative thereto) to force fluid to exit the openings rather than the distal tip of the sheath. In some embodiments, the fluid travels distally to exit one or more openings or ports at the distal end of the infusion sheath, which may include one or more slits or one or more holes or one or more elongated holes at a distal portion of the infusion sheath. In some embodiments, the fluid travels distally to exit the distal end of the sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location.

In some embodiments, the infusion sheath comprises two coaxial tubes forming a fluid column therebetween for the fluid to be delivered from the proximal end to a distal portion of the coaxial tubes. The infusion sheath is also coaxial with the longitudinal axis of the probe at the distal portion of the infusion sheath, at least. In some embodiments, the fluid travels distally to exit one or more openings or ports at the distal end of the infusion sheath, which may include one or more slits or one or more holes or one or more elongated holes at a distal portion of the infusion sheath. In some embodiments, the fluid travels distally to exit the distal end of the sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location.

In some embodiments, the infusion sheath comprises a first lumen that is coaxial with the longitudinal axis of the probe, and a second lumen having a second longitudinal axis extending substantially parallel to the longitudinal axis of the probe and/or the first lumen for infusion of fluid (i.e. the drug) into the target site. As previously noted, the target site can include target tissue or be at the treated area in instances where the probe is activated prior to the fluid infusion to the same co-located site. In some embodiments, after cellular lysis, fluid is infused through the second lumen of the infusion sheath to co-locate the drug infused at the target site without moving the energy probe, maintaining accuracy of the infusion location. The infusion sheath may have one or more openings or ports at its distal end in fluid communication with the second lumen through which the fluid exits the infusion sheath. The openings or ports may be slits, holes, elongated holes or other suitable shapes, and may be positioned to infuse the fluid substantially parallel to, coaxially with, or both, relative to the second longitudinal axis, normally to the second longitudinal axis, radially relative to the second longitudinal axis, or in any other combination or direction.

In some embodiments, the infusion sheath is a multi-lumen tube for infusion of fluid (i.e. the drug) into the target site, which can include target tissue or be at the treated area in instances where the probe is activated prior to the fluid infusion to the same co-located site. The multiple lumens may be parallel to a central lumen through which the probe extends. In some embodiments, after cellular lysis, fluid is infused through a lumen of the infusion sheath, or through the multiple lumens of the infusion sheath (in such embodiments) to co-locate the drug infused at the target site without moving the energy probe, maintaining accuracy of the infusion location.

In some embodiments, the energy probe is retractable back into the infusion sheath. In some embodiments, the energy probe extends out the distal end of the infusion sheath for placement. In some embodiments, after cellular lysis treatment is performed the probe is retracted into the infusion sheath far enough that infused fluid can move around the end of the retracted energy probe and out the end hole of the infusion sheath, maintaining accuracy of the infusion location. Relative movement of the probe and infusion sheath is possible in the embodiments herein. Thus, while retraction of the probe into a stationary infusion sheath may be mentioned, the embodiments herein may also functionally be used with the probe stationary and the sheath moving relative thereto. In order to co-locate the fluid (i.e. drug) where the energy probe withdrew or delivered its energy, where the sequence of treatment is energy delivery/withdrawal then drug delivery, it is the latter relative movement noted (i.e. probe stationary with sheath advancing distally over the probe to deliver fluid while advancing or after advancement) that may be more advantageous in some circumstances. Alternatively, however, it is envisioned that other locations of delivery of fluid or drug relative to the treatment site of energy delivery or withdrawal may be advantageous. In such instances, the embodiments described herein with co-axial delivery of the infusion sheath and its openings with the probe allows for optionality along with accuracy and control of the fluid delivery relative to the energy delivery location.

In some embodiments, the outer diameter of the infusion sheath may be 2 mm or larger. In some embodiments, the infusion sheath consists of a single or multichannel fluid path. In some embodiments, the infusion sheath may be a polymer such as PTFE, Nylon, PP, PE or metal such as stainless steel or nitinol. In some embodiments, the infusion sheath has a side arm with a tube for connecting to an infusion source, which may include a pump. In some embodiments, the infusion pump is any pump suited to transport fluids. In some embodiments, the infusion pump is a roller pump, syringe pump, syringe cartridge, or hand syringe. In some embodiments, the side arm tubing extends from the infusion sheath 30 cm to 245 cm. In some embodiments, the inner diameter of the side arm tubing is small and provides a low priming volume such that the volume required to prime the system is less than or equal to 0.01 mL/cm, or less than or equal to 0.005 mL/cm, or less than or equal to 0.0075 mL/cm, reducing potential waste of drug in the side arm tubing. This disclosure recognizes that the infusion of drug is not dependent on the type of drug being infused. In some embodiments, the drug is one or multiple drugs selected by the user that is best for the subject.

### Fluid Such as Therapeutic Agent

Traditional clinical approaches for cancer immunotherapy rely on the systemic administration of high doses of one or two biological immunomodulatory agents. Major advances in the treatment of cancer have been made in the last decade by the use of monoclonal antibodies (mAbs) that block immune checkpoint inhibitor molecules such as PD-1, PD-L1 and CTLA-4. However, despite the significant benefits obtained by the use of these mAbs, a substantial fraction of patients still do not respond to the therapy or become refractory to it. Additionally, the use of high systemic doses of these checkpoint inhibitors is accompanied by high frequency of serious adverse events, particularly when used in combination with other immunotherapeutic agents, which limits the number of agents and doses that can be combined to address the multifactorial nature of tumor immune resistance.

As described herein, any fluid may be delivered with the device. For example, the fluid may be a therapeutic agent. Any fluid may be a therapeutic agent. In some embodiments, the therapeutic agent is configured to treat cancer. For example, the therapeutic agent may be one, two, or more biological immunomodulatory agents.

Traditional cancer immunotherapeutic approaches only provide benefit to a limited fraction of patients. This may be due to systemic delivery of drugs which is limited in effectiveness and is associated with toxicity and adverse events. The methods, systems, and apparatuses provided and described herein deliver drugs intratumorally and as a result, the amount of drug required is reduced compared to systemic delivery. Intratumoral delivery immediately following cellular lysis can carry the intracellular components, i.e., tumor specific antigens (TSA), tumor associated antigens (TAA), damage associated molecular patterns (DAMPs) and other cellular components released by the breakdown of the cellular wall, into the tumor environment and lymphatic system thereby initiating an immune response. By infusing immediately post cell lysis and accurately in the center of the treatment zone the entrainment of the released antigens and other cellular components may be optimized. Additionally, intratumoral administration allows for a significant reduction in dose, which reduces the probability of toxicity and adverse events. Provided herein are methods, systems, and apparatuses that allow a reduced dose of drug to be delivered accurately into the target cellular lysis zone in order to effectively remove cellular debris from the tumor microenvironment of cellular components that has flowed into the area due to the cellular lysis treatment.

### Target Site

As used herein, the devices are suitable for treatment and co-location of energy delivery (such as cryo) and fluid (such as a drug) to a variety of target sites. The target sites may be target tissue such as cancer tissue or cancer or tumors comprising cancerous tissue. A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” may include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues, but it has not metastasized, except potentially to the lymph nodes. In other embodiments, the cancer or tumor is stage IV, such that, e.g., the cancer or tumor has metastasized.

In some embodiments, the cancer comprises adenocarcinoma, adrenal cortical cancer, anal cancer, astrocytoma, bile duct cancer, bladder cancer, bone cancer, bone sarcoma, brain tumors, brain cancer, breast cancer, cervical cancer, chordoma, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, glioblastoma, glioma, Hodgkin disease, Kaposi sarcoma, kidney cancer, renal cell carcinoma, laryngeal and hypopharyngeal cancer, liver cancer, hepatocellular carcinoma (HCC), non-small cell lung cancer, small cell lung cancer, lung carcinoid tumor, medulloblastoma, melanoma, meningioma, mesothelioma, metastatic pituitary carcinoma, nasal cavity and paranasal sinus cancer, nasopharyngeal cancer, neuroblastoma, non-Hodgkin lymphoma, non-melanoma skin cancer, oral cancer, ovarian cancer, oral cavity and oropharyngeal cancer, osteosarcoma, ovarian cancer, pancreatic cancer, penile cancer, pituitary tumors, prostate cancer, renal cell carcinoma, retinoblastoma, rhabdomyosarcoma, salivary gland cancer, sarcoma, small intestine cancer, stomach cancer, squamous cell carcinoma (including head and neck cancer), testicular cancer, throat cancer, thymus cancer, thyroid cancer, uterine sarcoma, vaginal cancer, Wilms tumor, and any combination thereof.

In some embodiments, a cancer is a solid tumor cancer. In some embodiments, a solid tumor cancer is selected from adenocarcinoma, astrocytoma, bladder cancer, bone sarcoma, breast cancer, cervical cancer, chordoma, colorectal cancer, endometrial cancer, esophageal cancer, glioblastoma, glioma, kidney cancer, liver cancer, medulloblastoma, melanoma, meningioma, mesothelioma, metastatic pituitary carcinoma, prostate cancer, neuroblastoma, non-melanoma skin cancer, non-small cell lung cancer, oral cancer, ovarian cancer, pancreatic cancer, renal cell carcinoma, retinoblastoma, sarcoma, small cell lung cancer, squamous cell carcinoma (including head and neck cancer), stomach cancer, testicular cancer, thyroid cancer, Wilms tumors, and any combination thereof. In some embodiments, a cancer is metastatic prostate cancer. In some embodiments, a cancer is metastatic breast cancer. In some embodiments, a cancer is non-small cell lung cancer.

### Systems, Methods, and Devices

As described here, an energy probe is used to either deliver energy to a treatment site, or withdraw energy from a treatment site, to cause cellular disruption at the treatment site. In a preferred embodiment, a cryolysis system is configured to slidably transition between two or more positions. In some embodiments, a first position is configured such that an infusion sheath distal end is spaced apart from a distal tip of an energy probe in the distal direction by a distance. In some embodiments, a second position is configured such that the infusion sheath distal end is spaced apart from the distal tip of the energy probe in the distal direction by a distance smaller than the distance in the first position. In some embodiments, a third position is configured such that the infusion sheath distal end is spaced apart from the distal tip of the energy probe at a distance greater than 1 cm, completely covering the probe's distal tip, and allowing infusible drug to flow past the distal tip through an interior lumen of the infusion sheath. In some embodiments, the third position is not included. In some embodiments, the third position is optional.

As used herein, the terms first, second, and third position are used to indicate a configuration of the cryolysis system and relative positions of the infusion sheath and distal tip of the probe at different points in time and are not meant to denote any specific sequencing of these positions or configurations. As used herein, the terms first and second position are used to indicate a configuration of the cryolysis system at a point in time and are not meant to denote any specific sequencing of these positions. For example, in a non-limiting example, the cryolysis system is inserted into a tissue in the second position, an infusion sheath is then retracted to the first position to allow energy to be applied to or be withdrawn from the tissue, and then the sheath is moved to the third position to allow infusible drug to flow into the tissue. Similarly, in another non-limiting example, the cryolysis system is inserted into the tissue in the first position, energy is applied to/withdrawn from the tissue via an energy probe, followed by the infusion sheath moving to the second position and further moving into the third position to allow drug to be infused. In another non-limiting example, the cryolysis system is inserted into the tissue in the first position where energy is applied to or is withdrawn from the tissues, then the infusion sheath is advanced distally to the second position where drug is infused into the tissue.

The systems, methods, and devices described herein are not limited to three positions. For example, the cryolysis system may have two positions, four positions, or any other number of potential positions. The cryolysis system may have a single position in the case that infusible drug could be released while maintaining the position of the infusion sheath, such as by rotating the infusion sheath about the longitudinal axis, allowing infusible drug to flow through the infusion sheath, past the energy probe, and into the tissue. The cryolysis system may allow for delivery of the drug while the infusion sheath is moving relative to the probe, or while the probe is moving relative to the infusion sheath.

For orientation, with respect to any particular element or embodiment disclosed below, unless the context makes it clear or it is explicitly stated otherwise, the term “proximal” usually means the portion of the particular element or embodiment closest to a user thereof, while “distal” usually means the portion of the particular element or embodiment furthers away from a user thereof.

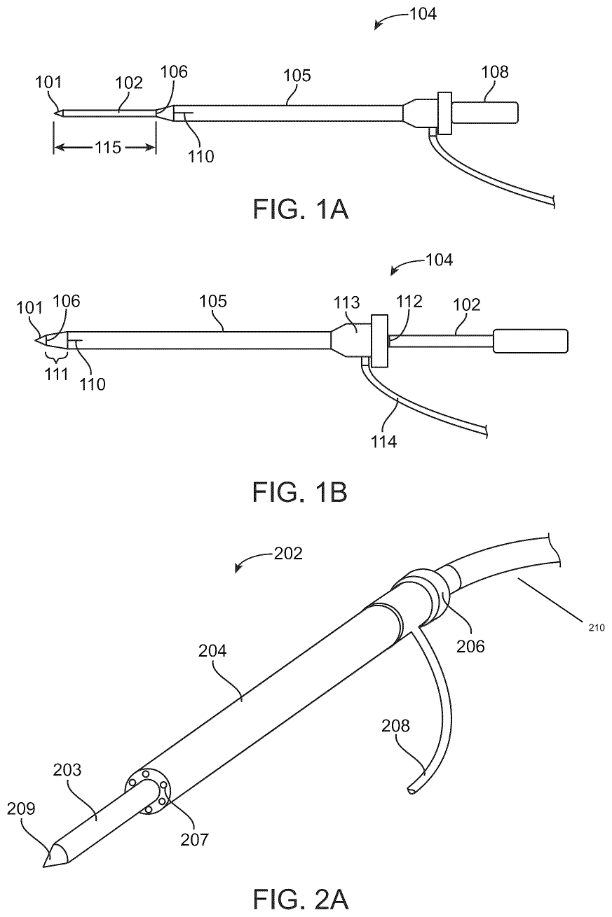

FIG. 1A illustrates an example of a cryolysis system 104 in a first position comprising probe 102 and an infusion sheath 105. When in the first position as shown in FIG. 1A, a distal tip 101 of the probe 102 extends to a first location which is distal to a second location of a sheath distal end 106. In some embodiments, the probe 102 is configured to emit energy to or to withdraw energy from the surrounding tissue when in the first position. For example, the probe 102 may freeze a plurality of cells in a target tissue, thereby causing cell lysis. In some embodiments, when in the first position, the infusion sheath 105 is configured to retain an infusion of therapeutic agent, to thereby prevent freezing of the therapeutic agent prior to delivery, during freezing of the target tissue by the probe tip. For example, the infusion sheath may be configured to fluidly couple with a reservoir containing the therapeutic agent, see for example element 114 in FIG. 1B, so as to deliver the therapeutic agent from the reservoir to the patient.

In some embodiments, the probe 102 is an energy probe. In some embodiments, the probe 102 is a cryoprobe. In some embodiments, a handle 108 is coupled with the proximal end of the probe 102, at its distal end, while its proximal end is coupled or couplable with a source of cryogen, a source to drive a Pulsed Electric Field energy probe, or the like. In some embodiments, the probe 102 extends to a distal tip 101. In some embodiments, the distal tip 101 is sharp. In some embodiments, the distal tip 101 is tapered up towards the distal end of the probe 102. In some embodiments, the distal tip 101 is configured to penetrate one or more different tissues in the human body. For example, the distal tip 101 may be configured to pass through skin and one or more tissues to reach the target site. In some embodiments, the distal tip 101 is round and atraumatic.

In some embodiments, the energy probe 102 extends through an infusion sheath 105. In some embodiments, the infusion sheath 105 comprises a port 110 extending through the outer surface of the infusion sheath 105 to the center lumen of the sheath 105. In some embodiments, the port 110 is a hole, slit, elongated hole, is circular, triangular, square, rectangular, pentagonal, heptagonal, hexagonal, or any other shape permitting flow of fluid out of the lumen of the infusion sheath 105 and to the exterior environment, which, as contemplated herein is a target site.

In some embodiments, the infusion sheath 105 is configured to be slidably moved along the longitudinal axis of the probe 102 such that a longitudinal axis of the probe 102 and a longitudinal axis of the infusion sheath 105 are coaxial.

In some embodiments, the distal end of the infusion sheath 105 has at least one port 110 disposed through the wall of the infusion sheath 105 to communicate with the inner diameter lumen of the infusion sheath 105. For example, the at least one port 110 may be configured to permit an egress point for fluids flowing through the infusion sheath 105. In some embodiments, the at least one port 110 is configured to be disposed through the infusion sheath 105 such that loss of axial strength of the infusion sheath 105 outer wall is minimized or prevented. In some embodiments, the at least one port 110 is configured to be disposed through the infusion sheath 105 such that no material has been removed from the infusion sheath 105 outer wall when the at least one port 110 was created.

In some embodiments, the port 110 is disposed through the outer surface of the infusion sheath 105 in a longitudinal direction. In some embodiments, the port 110 is disposed through the outer surface of the infusion sheath 105 radially. In some embodiments, the infusion sheath 105 comprises 1, 2, 3, 4, 5, 6, 7, 8, 9, 10, 11, 12, 13, 14, 15, 16, 18, 19, or 20 ports 110. In some embodiments, the infusion sheath 105 comprises four ports 110. In some embodiments, the infusion sheath 105 comprise two or more ports 110, where the ports 110 are distributed radial-symmetrically about the longitudinal axis of the infusion sheath 105.

In some embodiments, the distal tip 101 is spaced apart from the sheath distal end 106 in the first position by a distance 115. In some embodiments, the distance 115 is 1 cm, 2 cm, 3 cm, 4 cm, or 5 cm. In some embodiments, the distance 115 is 0 to 1 cm. In some embodiments, the distance 115 is 0 to 2 cm. In some embodiments, the distance 115 is 0 to 3 cm. In some embodiments, the distance 115 is 0 to 4 cm. In some embodiments, the distance 115 is 0 to 5 cm. In some embodiments, the distance 115 is 1 to 2 cm. In some embodiments, the distance 115 is 1 to 3 cm. In some embodiments, the distance 115 is 1 to 4 cm. In some embodiments, the distance 115 is 1 to 5 cm. In some embodiments, the distance 115 is 2 to 3 cm. In some embodiments, the distance 115 is 2 to 4 cm. In some embodiments, the distance 115 is 2 to 5 cm. In some embodiments, the distance 115 is 3 to 4 cm. In some embodiments, the distance 115 is 3 to 5 cm. In some embodiments, the distance 115 is 4 to 5 cm.

FIG. 1B illustrates an example of the cryolysis system 104 shown in FIG. 1A in a second position, according to embodiments described herein. When in the second position, the sheath distal end 106 and the distal tip 101 are spaced at a closer distance than in the first position. A source of cryogen is also provided but not shown in this figure for simplicity, as noted above, coupled or couplable with handle 108.

In some embodiments, the cryolysis system 104 is configured to slidably move between the first position and the second position. For example, the infusion sheath 105 may be configured to slidably move in a distal direction along a longitudinal axis of the probe 102 from the first position to the second position, where the second position is spaced apart from the first position. In some embodiments, the distal tip 101 is configured to slidably move in a proximal direction from the first position to the second portion. In some embodiments, the infusion sheath 105 is configured to release an infusion of a therapeutic agent when in the second position.

In some embodiments, the cryolysis system 104 is configured to transition between a third position and the second position. In some embodiments, when in the third position, the infusion sheath 105 covers the distal tip 101 or is positioned adjacent to the distal tip 101. For example, in this third position the probe 102 coupled with the infusion sheath 105 in the third position may be configured to prevent buckling of the infusion sheath and/or provide enhanced mechanical strength relative to the energy probe alone. For example, the enhanced mechanical strength may be provided about or adjacent to the distal end of the energy probe by the close positioning of the infusion sheath 105. In some embodiments, the cryolysis system 104 is configured to slidably move from the third position to the first position, thereby exposing the distal end 101 of the probe 102 for cell lysis.

In some embodiments, the infusion sheath 105 is slidably movable along the longitudinal axis relative to the probe 102. For example, the infusion sheath 105 may be moved in a distal direction relative to the longitudinal axis of the probe 102, thereby facilitating infusion of one or more fluids through the infusion sheath 105 and into a target site. In some embodiments, the infusion sheath 105 is configured to be moved in a distal direction relative to the longitudinal axis of the probe 102, to the target site after energy has been applied to or withdrawn from the target site to lyse cells.

In some embodiments, the at least one port 110 is disposed in the infusion sheath 105 at a location proximal to a tapered portion 111. In some embodiments, the at least one port 110 is disposed through at least a portion of the tapered portion 111.

In some embodiments, the advancement distance of the infusion sheath 105 is determined by moving the proximal end of the sheath to align with an indicator line 112 disposed on the outer surface of the probe 102. In some embodiments, the position of the sheath distal end 106 is located such that 0 to 2.5 cm of the distal end of the probe 102 is exposed before drug infusion is started. For example, the distal end of the probe 102 may be flush with the sheath distal end 106, thereby creating a distance of 0 cm between the distal end of the probe 102 and the sheath distal end 106.

In some embodiments, the infusion sheath 105 is aligned with the indicator line 112 disposed on the outer surface of the probe 102. In some embodiments, the distal tip 101 is exposed when the cryolysis system 104 is inserted into the target tissue thereby minimizing the risk for buckling. In some embodiments, the probe 102 provides or withdraws the energy necessary to create cellular lysis in the target tissue.

In some embodiments, after energy has been applied to or withdrawn from the target tissue and cellular lysis has occurred, the infusion sheath 105 is advanced forward along the longitudinal axis of the probe 102 until the valve 113 has passed by the indicator line 112 and the indicator line 112 is visible on the probe 102. In some embodiments, the indicator line 112 is inscribed into the probe 102. In some embodiments, the indicator line 112 is a raised surface or edge that creates a snap with the inner diameter of the valve 113 or an audible click when the valve 113 passes over the indicator line 112, alerting the user that the infusion sheath 105 is in the correct position. When the infusion sheath 105 is in the correct position, the at least one port 110 is positioned with the distal tip 101 of the probe 102 in the center of the target tissue.

When the infusion sheath 105 is in the correct position, drug infusion can be performed through the tube 114. In some embodiments, fluid is pumped through the tube 114 into the infusion sheath 105. In some embodiments, the valve 113 prevents any fluid from leaking out of the proximal end of the infusion sheath 105. The fluid travels down the infusion sheath 105 until it reaches the distal end of the infusion sheath 105, where it passes through the at least one port 110 and into the target tissue. In some embodiments, the material construction of the infusion sheath 105 is made of a flexible polymer. For example, the flexible polymer may be PTFE, PP, nylon, urethane, or FEP. In some embodiments, the material construction of the infusion she ath 105 is made of a rigid polymer. For example, the rigid polymer may be HDPE or polyamide. In some embodiments, the material construction of the infusion sheath 105 is metal to allow for insertion into dense target tissues such as bone. For example, the metal may be stainless steel, nitinol, or cobalt chromium. In some embodiments, the outer diameter of the infusion sheath 105 is 1.5 mm to 2.7 mm. In some embodiments, the outer diameter of the infusion sheath 105 is 1 mm to 4 mm.

In some embodiments, the outer surface of the infusion sheath 105 has a tapered portion 111 extending between the infusion sheath 105 and the distal tip 101. For, example, a narrow end of the tapered portion 111 may interface with the distal tip 101 while a second end on the tapered portion 111 having a diameter wider than the narrow end interfaces with the infusion sheath 105 portion extending from the valve 113.

In some embodiments, the infusion sheath 105 has a valve 113 coupled to the proximal end of the infusion sheath 105. In some embodiments, the valve 113 is configured to allow fluid to flow in the infusion sheath from the proximal end of the probe 102 to the distal end of the probe 102 and prevent fluid flow in reverse. In some embodiments, the valve 113 is a hemostasis valve.

In some embodiments, a tube 114 is connected to the side of the infusion sheath 105. In some embodiments, the tube 114 is configured to allow for the introduction of fluids from an infusion pump into the infusion sheath 105. In some embodiments, the fluid being introduced can come from any type of infusion pump or syringe (not shown).

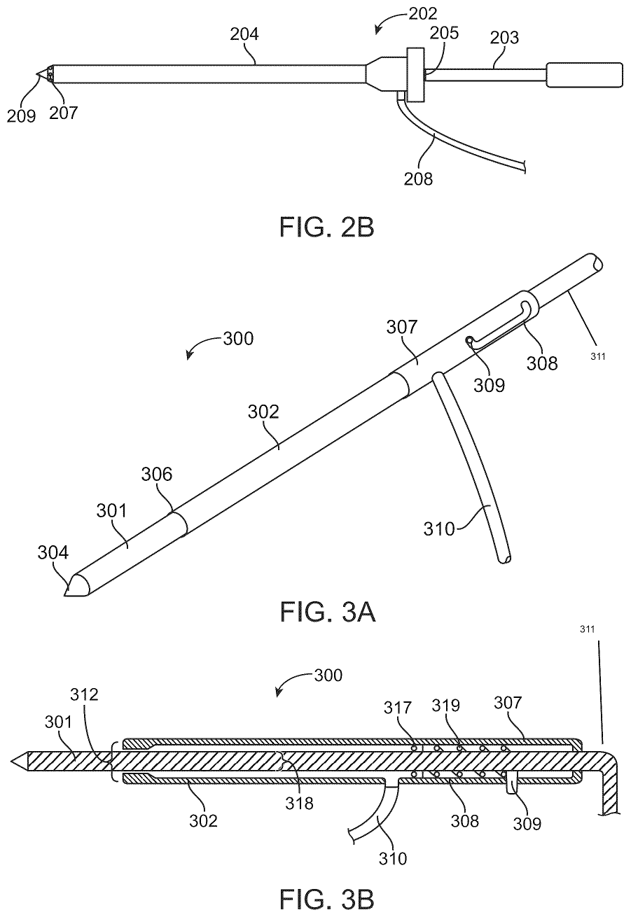

FIG. 2A illustrates an example of a cryolysis system 202 comprising a probe 203 and an infusion sheath 204 in a first position. In some embodiments, the probe 203 is an energy probe. In some embodiments, the probe 203 is a cryoprobe.

In some embodiments, the infusion sheath 204 is coaxial with and slidably movable along the longitudinal axis relative to the probe 203. For example, the infusion sheath 204 may be moved from the first position in a distal direction relative to the longitudinal axis of the probe 203 to a second position (see FIG. 2B), thereby facilitating infusion of one or more fluids through the infusion sheath 204 and into a target site defined by the energy probe tip 209. In some embodiments, the infusion sheath 204 is configured to be moved in a distal direction relative to the longitudinal axis of the probe 203, to the target site after energy has been applied to or withdrawn from the target site to lyse cells. In some embodiments, the position of the sheath distal end is located such that 0 to 2.5 cm of the distal end of the probe 203 is exposed before drug infusion is started. For example, the distal end of the probe 203 may be flush with the sheath distal end, thereby creating a distance of 0 cm between the distal end of the probe 203 and the sheath distal end.

In some embodiments, the cryolysis system 202 is configured to transition between the first position and the second position by retracting the probe 203 from the first position in a proximal direction relative to the longitudinal axis of the infusion sheath 204 to reach the second position where an infusion of therapeutic agent is released. In some embodiments, the advancement distance of the infusion sheath 204 is determined by moving the proximal end of the sheath to align with an indicator line 205 disposed on the outer surface of the probe 203. In some embodiments, the cryolysis system 202 is configured to transition between a third position and the first position, as described in reference to FIG. 1A and FIG. 1B.

In some embodiments, the infusion sheath 204 has a valve 206 coupled to the proximal end of the infusion sheath 204 and to probe 203 enclosed within the infusion sheath, at the distal end of valve 206, while at the proximal end thereof, valve or coupling 206 is also coupled at the proximal end thereof, to a tube 210 or the like as a source of cryogen or other source of energy to activate the energy probe. In some embodiments, the valve 206 is configured to allow fluid to flow in the infusion sheath 204 from the proximal end of the probe 203 to the distal end of the probe 203 and prevent fluid flow in reverse. In some embodiments, the valve 206 is a hemostasis valve. In some embodiments, end holes 207 are configured to be at the distal end of the infusion sheath 204. In some embodiments, there are a plurality of end holes 207 configured to be at the distal end of the infusion sheath 204.

In some embodiments, a tube 208 is connected to the side of the infusion sheath 204. In some embodiments, the tube 208 is configured to allow for the introduction of fluids from an infusion pump into the infusion sheath 204. In some embodiments, the fluid being introduced can come from any type of infusion pump or syringe (not shown).

In some embodiments, the probe 203 extends to a distal tip 209. In some embodiments, the distal tip 209 is sharp. In some embodiments, the distal tip 209 is round and atraumatic. In some embodiments, the distal tip 209 is tapered towards the distal end of the probe 203. In some embodiments, the distal tip 209 is configured to penetrate one or more tissues in the human body. For example, the distal tip 209 may be configured to pass through skin and one or more tissues to reach the target site. In some embodiments, the infusion sheath 204 is a multichannel tube in fluid communication with the tube 208. In some embodiments, the channels of the infusion sheath 204 run in the direction of the longitudinal axis of the infusion sheath 204. In some embodiments, the channels of the infusion sheath 204 transport fluids from the tube 208 down the longitudinal axis of the infusion sheath 204 and deliver the fluid to end holes 207.

FIG. 2B illustrates an example of the cryolysis system 202 shown in FIG. 2A in a second position, without showing the cryogen source, as shown in FIG. 2A, for simplicity, as noted above in relation to FIG. 1A and FIG. 1B. When in the second position, a sheath distal end and the distal tip 209 are spaced at a closer distance than in the first position. The position of the sheath distal end is located such that 0 to 2.5 cm of the distal end of the probe 203 is exposed before drug infusion is started. For example, the distal end of the probe 203 may be flush with the sheath distal end, thereby creating a distance of 0 cm between the distal end of the probe 203 and the sheath distal end.

In some embodiments, the infusion sheath 204 is configured to be moved along the longitudinal axis of the probe 203 such that the infusion sheath 204 is aligned with the indicator line 205. In some embodiments, the indicator line 205 is disposed on the outer surface of the probe 203 and the distal tip 209 is exposed. The cryolysis system 202 may be inserted into the target tissue without the potential for buckling. In some embodiments, the probe 203 provides or withdraws the energy necessary to create cellular lysis in the target tissue.

In some embodiments, after energy has been applied to or withdrawn from the target tissue and cellular lysis has occurred, the infusion sheath 204 is advanced forward along the longitudinal axis of the probe 203 until the valve 206 has passed by the indicator line 205 and the indicator line 205 is visible on the probe 203. In some embodiments, the indicator line 205 is inscribed into the probe 203. In some embodiments, the indicator line 205 is a raised surface or edge that creates a snap with the inner diameter of the valve 206 or an audible click when the valve 206 passes over the indicator line 205, alerting the user that the infusion sheath 204 is in the correct position. When the infusion sheath 204 is in the correct position, the end holes 207 are positioned with the distal tip 209 of the probe 203 in the center of the target tissue.

When the infusion sheath 204 is in the correct position, drug infusion can be performed through the tube 208. In some embodiments, fluid is pumped through the tube 208 into the infusion sheath 204. In some embodiments, the valve 206 prevents any fluid from leaking out of the proximal end of the infusion sheath 204. The fluid travels down the infusion sheath 204 until it reaches the distal end of the infusion sheath 204, where it passes through the end holes 207 and into the target tissue. In some embodiments, the material construction of the infusion sheath 204 is made of a flexible polymer. For example, the flexible polymer may be PTFE, PP, nylon, urethane, or FEP. In some embodiments, the material construction of the infusion sheath 204 is made of a rigid polymer. For example, the rigid polymer may be HDPE or polyamide. In some embodiments, the material construction of the infusion sheath 204 is metal to allow for insertion into dense target tissues like bone. For example, the metal may be stainless steel, nitinol, or cobalt chromium. In some embodiments, the outer diameter of the infusion sheath 204 is 1.5 mm to 2.7 mm. In some embodiments, the outer diameter of infusion sheath 204 is 1.0 mm to 4.0 mm.

FIG. 3A illustrates another embodiment of a cryolysis system 300 comprising a probe 301 and an infusion sheath 302 in a first position. In some embodiments, when in the first position, a probe distal end 304 extends distally from a distal end of the infusible sheath 306.

In some embodiments, the cryolysis system 300 comprises a probe 301, an infusion sheath 302, and a housing 307. In some embodiments, the probe 301 is an energy probe. In some embodiments, the probe 301 is a cryoprobe. In some embodiments, the housing 307 is attached to the proximal end of the infusion sheath 302. In some embodiments, the infusion sheath 302 is configured to move slidably along the longitudinal axis of the probe 301.

In some embodiments, the housing 307 is configured with a guide slot 308 that allows the housing 307 to move about a slide 309. In some embodiments, the slide 309 is attached to the probe 301. In some embodiments, the slide 309 is used to position the housing 307 and infusion sheath 302 such that the distal end of the probe 301 is exposed or covered by the infusion sheath 302. In some embodiments, the slide 309 or the guide slot 308 is disposed in a handle 307 of the probe 301. In some embodiments, the slide 309 is attached to the housing 307. In some embodiments, the guide slot 308 is disposed in the probe 301. In some embodiments, the slide 309 attached to the housing 307 is configured to slidably move along the guide slot 308 disposed in or on the probe 301.

In some embodiments, a tube 310 is configured to pass through the outer wall of the housing 307. In some embodiments, the tube 310 is configured to infuse fluid, such as an infusion of therapeutic agent into the infusion sheath 302, and a source of cryogen for a cryoprobe is provided at 311, with coupling occurring inside the handle 307.

FIG. 3B illustrates a cutaway profile view of the cryolysis system 300 in the first position.

In some embodiments, the cryolysis system 300 is left in the target tissue after cellular lysis has been created. In some embodiments, the probe 301 is retracted from the target tissue by sliding the slide 309 along the guide slot 308 from the distal position to a proximal position. In some embodiments, the probe 301 is slidably moved distally towards the target tissue by sliding the slide 309 along the guide slot 308 from the proximal position to the distal position. In preferred embodiments, as further described herein below, the sheath is advanced with respect to the probe 301 to permit infusion at the target site treated by the probe. In various embodiments defined herein when in use for treatment of a tumor, the device is sometimes referred to herein as an Oncolysis Infusion Device, or OID, to include the integration of the system for cellular fracture, preferably a cryolysis needle or probe, (but it may be any form of oncolysis probe), with an infusion system, as shown) for delivery of, for example, immunomodulatory agents at the target site. The source of cryogen 311 is coupled to the cryoprobe inside handle 307.

In some embodiments, when the slide 309 reaches the proximal position on the guide slot 308, the slide 309 is moved into the side branch of the guide slot 308 and clicks into place, alerting the user that the probe 301 has been fully retracted from the target tissue and into the infusion sheath 302. In some embodiments, the distal end of the infusion sheath 302 contains a taper 311 that increases the second inner diameter 312 along the longitudinal axis of the infusion sheath 302 towards to the proximal end such that when the probe 301 is retracted there is enough space between the second inner diameter 312 of the infusion sheath 302 and the outer diameter of the probe 301 to allow for fluid flow 313 past the distal tip of the probe 301.

In some embodiments, as shown in FIG. 3C, fluid flow 313 is introduced to the infusion sheath 302 through the tube 310. In some embodiments, fluid flow 313 travels down the length of the infusion sheath 302 and exits at the distal end of the infusion sheath 302 into the target tissue after the probe 301 has been retracted.

FIG. 3C illustrates an example of a cutaway view of the distal tip of the cryolysis system 300 in a second position. As shown in FIG. 3C, the probe 301 is entirely within the infusion sheath 302 when in the second position. In some embodiments, the probe 301 is slidably moved in a proximal direction relative to the longitudinal axis of the infusion sheath 302 thereby transitioning from the first position to the second position. In some embodiments, the infusion sheath 302 is slidably moved in a distal direction relative to the longitudinal axis of the probe 301 such that the infusion sheath distal end 314 is located distal to the probe distal end 316. In some embodiments, the cryolysis system 300 is configured to transition between a third position and the first position, as described in reference to FIG. 1A and FIG. 1B.

In some embodiments, the housing 307 contains a member 317 that acts as a retaining ring and seal at the proximal end of the housing 307. In some embodiments, the member 317 is configured to prevent fluids infused through the tube 310 from leaking out the proximal end of the infusion sheath 302. For example, the infusion sheath 302 may have a first inner diameter 318 and a second inner diameter 312 that is smaller than and located distal to the first inner diameter 318.

In some embodiments, the housing 307 contains a spring 319 inside the housing 307. In some embodiments, the spring 319 is coiled around the probe 301. In some embodiments, the spring 319 maintains the position of the probe 301 relative to the housing 307 when the slide 309 is moved either forward or backwards to advance or retract the sheath 302. When the slide 309 is moved into the forward position, the distal end of the probe 301 protrudes from the infusion sheath 302 at a distance distal to the distal end of the infusible sheath 306. In some embodiments, when the slide 309 is moved into the backwards position, the distal end of the probe 301 is covered by the infusion sheath 302.

In some embodiments, the outer diameter of the probe 301 is configured to be a near line to line fit with a second inner diameter 312 of the infusion sheath 302. In no event, however, does the infusion sheath 302 prevent the intentional exposure of the distal end of probe 312 so as to enable contact with the target tissue. In some embodiments, the partial protrusion of the probe 301 into the distal end 314 of the sheath 302 which creates a seal with the second inner diameter 312 of the infusion sheath 302. In this position, the cryolysis system 300 may be inserted into a target tissue by intentional protrusion of up to two centimeters beyond the terminal end 314 of the sheath 302. The length of the energy probe defines how deeply into a subject the probe component of the system has to penetrate in order to effect at least partial lysis within a targeted treatment zone. Once in place, with the tip 304, (as in 209, and in 101, in the above-described embodiments), the probe 301 may be activated to deliver or withdraw energy and create cellular lysis in the target tissue.

In some embodiments, the infusion sheath 302 having a first inner diameter 318 located at a proximal end of the infusion sheath and second inner diameter 312 located at a distal end of the infusion sheath. In some embodiments, first inner diameter 318 extends to a taper 311 located at a location between the distal end of the infusion sheath 302 and the proximal end of the infusion sheath 302 connecting the first inner diameter 318 region with the second inner diameter 312 region of the lumen of the infusion sheath 302. In some embodiments, the first inner diameter 318 is larger than the second inner diameter 312, to thus permit the placement of the distal end 316 of the probe 301 so as to form a scal with the distal end 314 of the infusion sheath 302, which prevents infusion flow 313, by blocking the lumen at the terminal end of the sheath 302.

In some embodiments, the second inner diameter 312 is substantially the same as the outer diameter of the probe 301. For example, when a portion of the distal end of the probe 301 is disposed within the second inner diameter 312, fluid does not flow from the inner lumen of the infusion sheath 302 through the distal end 314 of the infusion sheath 302. In some embodiments, the first inner diameter 318 is larger than the outer diameter of the probe 301. For example, when the probe 301 is at least partially disposed only in the portion of the sheath 302 having the first inner diameter 318 and not in the second inner diameter 312, fluid in the inner lumen of the infusion sheath 302 fluid flow 313 exits the distal end of the infusion sheath 302.

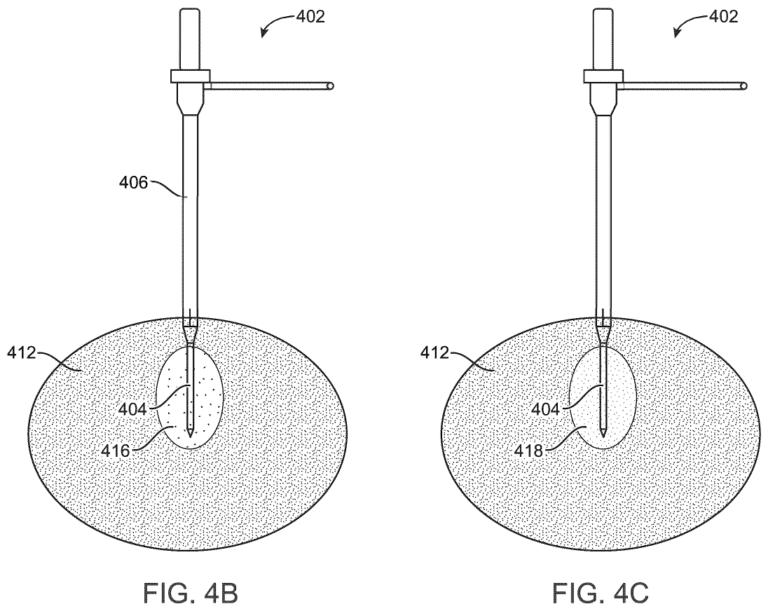

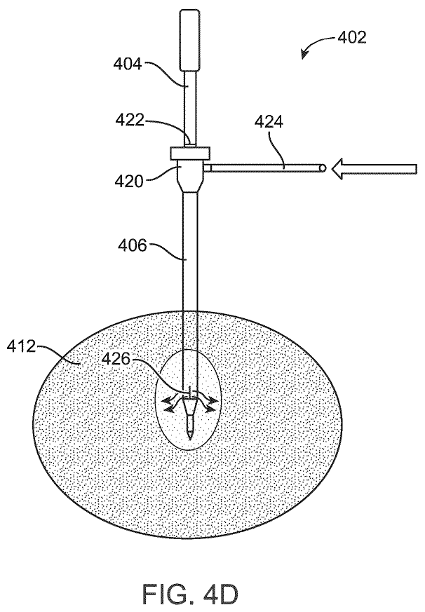

FIG. 4A-FIG. 4D illustrate a method for treating a target tissue by co-locally delivering or withdrawing energy and an infusion of therapeutic agent, and systems and methods thereof.

FIG. 4A illustrates an example of a cryolysis system 402 comprising a probe 404 and an infusion sheath 406 in a first position, according to embodiments described herein. When in the first position the distal tip 408 extends distally from the sheath distal end 410. In some embodiments, the distance between the distal tip 408 and the sheath distal end 410 is 1 cm, 2 cm, 3 cm, 4 cm, or 5 cm. In some embodiments, the distance between the distal tip 408 and the sheath distal end 410 is 0 cm to 1 cm. In some embodiments, the distance between the distal tip 408 and the sheath distal end 410 is 0 cm to 2 cm. In some embodiments, the distance between the distal tip 408 and the sheath distal end 410 is 0 cm to 3 cm. In some embodiments, the distance between the distal tip 408 and the sheath distal end 410 is 0 cm to 4 cm. In some embodiments, the distance between the distal tip 408 and the sheath distal end 410 is 0 cm to 5 cm. In some embodiments, the distance between the distal tip 408 and the sheath distal end 410 is 1 cm to 2 cm. In some embodiments, the distance between the distal tip 408 and the sheath distal end 410 is 1 cm to 3 cm. In some embodiments, the distance between the distal tip 408 and the sheath distal end 410 is 1 cm to 4 cm. In some embodiments, the distance between the distal tip 408 and the sheath distal end 410 is 1 cm to 5 cm. In some embodiments, the distance between the distal tip 408 and the sheath distal end 410 is 2 cm to 3 cm. In some embodiments, the distance between the distal tip 408 and the sheath distal end 410 is 2 cm to 4 cm. In some embodiments, the distance between the distal tip 408 and the sheath distal end 410 is 2 cm to 5 cm. In some embodiments, the distance between the distal tip 408 and the sheath distal end 410 is 3 cm to 4 cm. In some embodiments, the distance between the distal tip 408 and the sheath distal end 410 is 3 cm to 5 cm. In some embodiments, the distance between the distal tip 408 and the sheath distal end 410 is 4 cm to 5 cm.

In some embodiments, the method includes identifying a target tissue 412 for treatment. In some embodiments, the target tissue is a cancerous tumor. In some embodiments, the method includes disposing a portion of the cryolysis system 402 into the target tissue 412 such that the distal tip 408 of the probe 404 is within the target tissue 412. In some embodiments, at least a portion of the probe 404 and at least a portion of the infusion sheath 406 are disposed in the target tissue 412. In some embodiments, the probe 404 comprises a handle 414 located at the proximal end of the probe 404 to which a source of cryogen is coupled (not shown). In some embodiments, the cryolysis system 402 would be inserted into the target tissue 412 in this position. In some embodiments, the probe 404 is a cryoprobe.

In some embodiments, the method includes the steps of disposing a portion of a cryolysis system 402 comprising probe 404 coupled with an infusion sheath 406 at least partially into a target tissue 412 such that a distal tip 408 of the probe 404 is disposed in the target tissue 412. In some embodiments, the cryolysis system 402 is in the first position, a second position, or a third position when disposing in the target tissue 412. In some embodiments, the cryolysis system 402 is configured to transition between a third position and the first position, as described in reference to FIG. 1A and FIG. 1B.

In some embodiments, the method includes disposing a probe 404 of a cryolysis system 402 at least partially into a target tissue 412 of a target site such that a distal tip 408 of the probe 404 is disposed in the target tissue 412. An infusion sheath 406 of the system 402 may be positioned proximal to probe and proximal to the target tissue, and then be advanced distally to the target site and infuse the fluid into the target site and, therefore, the into target tissue 412. In some embodiments the infusion sheath may substantially bathe the target tissue in the fluid such that the fluid extends beyond the edges of the target tissue (i.e. tumor).

FIG. 4B illustrates an example of a step in the method for treating a target tissue by co-locally delivering or withdrawing energy and an infusion of therapeutic agent with the cryolysis system 402 where at least a portion of the target tissue 412 is lysed by energy applied or withdrawn by the probe 404. In some embodiments, once in position, the probe 404 is ready to deliver or withdraw energy to or from the target tissue 412 in order to impart cellular lysis. In some embodiments, cryogenic fluid, the source of which is coupled to 414, is delivered to the probe 404 to create a zone of cellular lysis 416. In some embodiments, the method includes the step of providing or withdrawing energy from the probe 404 to the target tissue 412 thereby lysing at least a portion of a plurality of cells in the target tissue 412.

FIG. 4C illustrates an example of a step in the method for treating a target tissue by co-locally delivering or withdrawing energy and an infusion of therapeutic agent with the cryolysis system 402 where the area frozen by the probe 404 forming the zone of cellular lysis 416 is thawed into a zone of lysed cells 418. In some embodiments, after the zone of cellular lysis 412 has been established within the target tissue 412 and the ice ball has totally dissipated, a zone of lysed cells 418 is created.

FIG. 4D illustrates an example of a step in the method for treating a target tissue by co-locally delivering or withdrawing energy and an infusion of therapeutic agent with the cryolysis system 402 where the cryolysis system 402 has transitioned to a second position. In some embodiments, the method includes slidably moving the infusion sheath 406 in a distal direction along a longitudinal axis of the probe 404 from the first position to the second position, wherein the second position is spaced apart from the first position. In some embodiments, the distance between the distal tip 408 and the sheath distal end 410 is 1 cm, 2 cm, 3 cm, 4 cm, or 5 cm. In some embodiments, the distance between the distal tip 408 and the sheath distal end 410 is 0 cm to 1 cm. In some embodiments, the distance between the distal tip 408 and the sheath distal end 410 is 0 cm to 2 cm. In some embodiments, the distance between the distal tip 408 and the sheath distal end 410 is 0 cm to 3 cm. In some embodiments, the distance between the distal tip 408 and the sheath distal end 410 is 0 cm to 4 cm. In some embodiments, the distance between the distal tip 408 and the sheath distal end 410 is 0 cm to 5 cm. In some embodiments, the distance between the distal tip 408 and the sheath distal end 410 is 1 cm to 2 cm. In some embodiments, the distance between the distal tip 408 and the sheath distal end 410 is 1 cm to 3 cm. In some embodiments, the distance between the distal tip 408 and the sheath distal end 410 is 1 cm to 4 cm. In some embodiments, the distance between the distal tip 408 and the sheath distal end 410 is 1 cm to 5 cm. In some embodiments, the distance between the distal tip 408 and the sheath distal end 410 is 2 cm to 3 cm. In some embodiments, the distance between the distal tip 408 and the sheath distal end 410 is 2 cm to 4 cm. In some embodiments, the distance between the distal tip 408 and the sheath distal end 410 is 2 cm to 5 cm. In some embodiments, the distance between the distal tip 408 and the sheath distal end 410 is 3 cm to 4 cm. In some embodiments, the distance between the distal tip 408 and the sheath distal end 410 is 3 cm to 5 cm. In some embodiments, the distance between the distal tip 408 and the sheath distal end 410 is 4 cm to 5 cm. In some embodiments, the method includes the steps of delivering an infusion of a therapeutic agent to the target tissue 412 from the infusion sheath 406. In some embodiments, the method includes the steps of removing the probe 404 from the target tissue 412 prior to delivering the fluid (i.e. the therapeutic agent) to the target tissue 412.

In some embodiments, the infusion sheath 406 is advanced forward such that the proximal end of the valve 420 is in line with the indicator line 422 on the probe 404 surface. In some embodiments, the probe 404 remains in place and acts as a guide for the infusion sheath 406 to be placed in the same location as probe 404 and the zone of lysed cells 418. In some embodiments, the side tube 424 of the infusion sheath 406 is connected to a syringe or other reservoir that has been filled with an infusion of a therapeutic agent i.e., one or more immunotherapeutic drugs. In some embodiments, the total volume of infusion of the therapeutic agent is 15 mL. In some embodiments, the total volume of infusion of the therapeutic agent is between 5 mL to −10 mL, 10 mL to 15 mL, 15 mL to 20 mL, or 20 mL to 25 mL. In some embodiments, the total volume of infusion of the therapeutic agent is between 1 mL to 10 mL, 10 mL to 20 mL, or 20 mL to 30 mL. In some embodiments, the syringe containing the drug is installed into a syringe pump and the system is primed with drug by advancing the syringe pump until drug fills the side tube 424.

In some embodiments, the infusion syringe pump is set to a flow rate of 3 mL/min. The pump is then started with the entire volume of 15 ml being infused in 5 minutes. In some embodiments, the valve 420 at the proximal end of the infusion sheath 406 prevents infused drug from leaking out of the proximal end of the infusion sheath 406. This is also prevented by halting fluid pressure in the line 424, which is connected to a pressure head or pump. In some embodiments, the infusion is directed to flow out of one or more slits 426 in the distal end of the infusion sheath 406. In some embodiments, the valve 420 is configured to keep the infusion sheath 406 in position, not allowing it to move along the longitudinal axis of probe 404, in readiness for delivery of infusion to the sheath 406.

In some embodiments, when the infusion pump has completed its infusion cycle and delivered the, for example, 15 mL out of the syringe, the syringe is disconnected and the side tube 424 is flushed with saline to infuse the remaining drug in the side tube 424 into the zone of lysed cells 418. In some embodiments, the volume of therapeutic agent is sufficient to both prime the infusion pump and deliver remaining volume of therapeutic agent to the zone of lysed cells 418 without flushing the side tube 424 with saline to infuse the remaining drug.

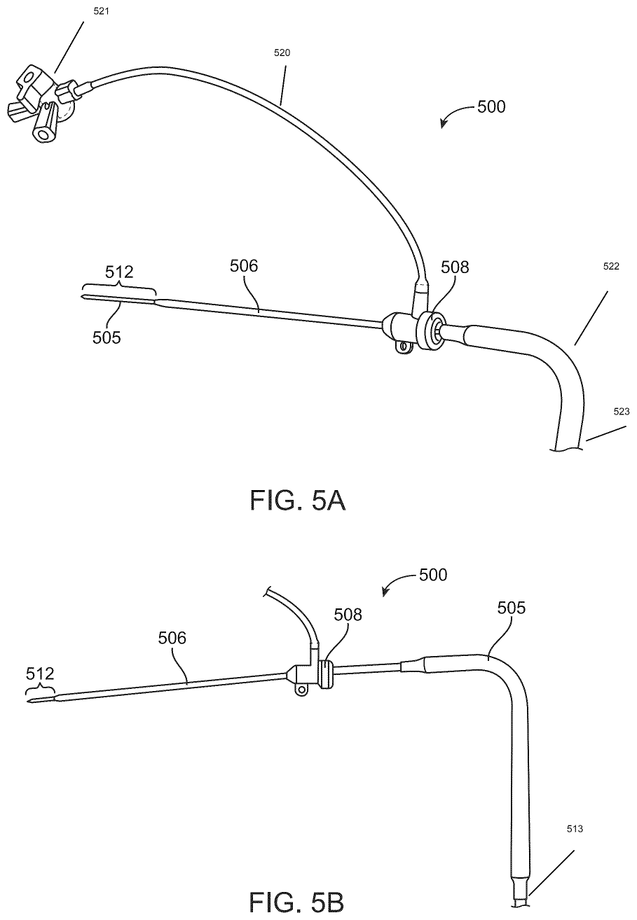

FIG. 5A illustrates an example of a cryolysis system 500 comprising a cryoprobe 505 and an infusion sheath 506 in a first position, according to embodiments described herein. In some embodiments, the cryolysis system 500 is made from off the shelf components or are purpose built as further described herein below. In some embodiments, the cryolysis system 500 comprises an infusion sheath 506, a cryoprobe 505, and a valve 508, at which point the cryoprobe internal lumen is coupled with a source of cryogen, or, alternatively, such coupling is provided at 523 in an angled cryoprobe, as shown in FIG. 5B. In some embodiments, the valve 508 is a hemostasis valve. In some embodiments, there is a cryoprobe tip 512 at the distal end of the cryoprobe 505 that can be inserted into a target tissue. In some embodiments, the infusion sheath 506 and the cryoprobe tip 512 are configured to have a snug fit at the location where the two meet, preventing any tissue from getting caught between the infusion sheath 506 and cryoprobe 505 when the cryolysis system 500 is inserted into the target tissue.

FIG. 5B illustrates a second view of the cryolysis system 500 shown in FIG. 5A in a second position, according to embodiments described herein. In some embodiments, the infusion sheath 506 is slidably moved in a distal direction along the longitudinal axis of the cryoprobe 505 towards the target tissue such that distance between the distal end of the infusion sheath 506 and the cryoprobe 505 is smaller than in the first position. In some embodiments, the cryoprobe 505 is slidably moved in a proximal direction relative to the longitudinal axis of the cryoprobe 505 away from the target tissue to move the cryolysis system 500 from the first position to the second position. In some embodiments, the cryoprobe 505 is completely retracted or covered by the sheath 506. For example, the cryoprobe 505 may be retracted after cryolysis of a portion of the target tissue and before an infusion of therapeutic agent is delivered through sheath 506.

In some embodiments, when the cryoprobe 505 is retracted from the infusion sheath 506 fluid can be infused into the target tissue after cryolysis has been performed and the infusion sheath 506 has been advanced to the second position of the cryoprobe 505. In some embodiments, the infusion sheath 506 has holes or slits at the distal end to allow fluid to exit the cryolysis system 500 into the target tissue. In some embodiments, the valve 508 prevents fluid from leaking out the proximal end of the infusion sheath 506. In this embodiment, the infusion tube 520 extends to a luer type valve 521 for connection to the source of infusion. Tube 522 is connected with the proximal end of cryoprobe 505 which is coupled with a source of cryogen 523.

In some embodiments, the infusion sheath 506 comprises an elongate, flexible tubular body, having a proximal end, a distal end and a central lumen. In some embodiments, the valve 508 is provided in a housing on the proximal end of the infusion sheath 506. In some embodiments, the valve 508 comprises a collapsible tubular sidewall defining a valve lumen in communication with the central lumen of the infusion sheath 506.

In some embodiments, the inside diameter of the tubing maybe be continuously controlled from the collapsible valve lumen's original fully opened inside diameter to fully closed, sufficiently to clinically eliminate leakage of fluid flow through the valve 508 with or without a dilator, guide wire(s), and/or a catheter in the valve lumen. This enables the valve 508 to function with no leaking of air or liquid at any state between fully closed and fully open as needed. In addition or alternatively, the source of cryogen 513 may be isolated by valve or coupling 508 or such control may be achieved at the source of cryogen 513.

In some embodiments, the valve 508 is shipped with a retention feature such as a pin or clip to keep the valve 508 open between production and use. In some embodiments, it is desirable to hold the valve 508 open for one or more steps such as to reduce friction with the cryoprobe 505 as it is being advanced or retracted through the valve 508. A retention feature or clip or control on the housing, which may be in the form of a handle, may be provided to permit selective, temporary locking of the valve 508 open during the procedure as may be desired.

### SELECTED EMBODIMENTS

Embodiment 1: A system for collocating an energy for cell lysis and an infusion of a fluid in a target site including tissue of a subject, the system comprising:
1. an energy probe configured to emit or withdraw an energy into or from the tissue of the target site thereby causing cell lysis of said tissue; and
2. an infusion sheath having a lumen configured to at least partially surround the energy probe, wherein the infusion sheath is configured to provide a pathway for the fluid to travel distally within the lumen and exit a distal portion of the infusion sheath, wherein the probe is coaxial with the infusion sheath and shares a longitudinal axis, thereby allowing co-location of the energy delivered from or withdrawn by the probe and the infusion of the fluid from the infusion sheath in the target site.

Embodiment 2: The system according to embodiment 1, wherein the infusion sheath comprises at least one port in the distal portion of the infusion sheath that is in fluid communication with the pathway of the infusion sheath.

Embodiment 3: The system according to embodiment 1 or 2, wherein the at least one port is a slit.

Embodiment 4: The system according to any one of embodiments 1 to 3, wherein the energy probe and the infusion sheath are translatable relative to one another along the longitudinal axis.

Embodiment 5: The system according to any one of embodiments 1 to 4, wherein the pathway and port are configured to deliver the fluid while the probe is within the pathway at the distal end of the infusion sheath.

Embodiment 6: The system according to any one of embodiments 1 to 5, comprising a valve configured to eliminate proximal flow of the fluid during and/or following delivery thereof to the target site.

Embodiment 7: The system according to any one of embodiments 1 to 6, comprising a tube in fluid communication with the pathway of the infusion sheath at a distal end of the tube and in fluid communication with a source of the fluid at a proximal end of the tube.

Embodiment 8: The system according to any one of embodiments 1 to 7, wherein the fluid is a therapeutic agent effective in treating the tissue at the target site.

Embodiment 9: The system according to embodiment 8, wherein the therapeutic agent is a cancer therapeutic agent.

Embodiment 10: The system according to any one of embodiments 1 to 9, wherein the energy exchange induces cryolysis of tissue at the target site.

Embodiment 11: The system according to any one of embodiments 1 to 10, comprising a location indicator configured to provide a visual, tactile, or audible indication of the probe's position relative to the infusion sheath's position or vice versa.

Embodiment 12: The system according to any one of embodiments 1 to 11, comprising a location indicator configured to provide a visual, tactile, or audible indication of a position of a port of the infusion sheath relative to the probe's position, or vice versa.

Embodiment 13: The system according to any one of embodiments 1 to 12, comprising a channel disposed in the exterior surface of the energy probe, the channel configured to slidably couple with a protrusion extending from the interior wall of the infusion sheath, thereby preventing circumferential rotation of the infusion sheath relative to the energy probe.

Embodiment 14: The system according to any one of embodiments 1 to 13, comprising a channel disposed at least partially through a wall of the infusion sheath, the channel configured to slidably couple with a protrusion extending from the exterior surface of the energy probe, thereby preventing circumferential rotation of the infusion sheath relative to the energy probe.

Embodiment 15: The system according to any one of embodiments 1 to 14, comprising a valve configured to connect an infusion tube with a proximal end of the infusion sheath, so as to provide the infusion of the fluid to the enter lumen of the infusion sheath and/or a plurality of fluid channels disposed within the walls of the sheath.

Embodiment 16: The system according to any one of embodiments 1 to 15, wherein the infusion sheath has a first inner diameter located at a proximal end of the infusion sheath and a second inner diameter located at a distal end of the infusion sheath.

Embodiment 17: The system according to embodiment 16, wherein the first inner diameter is larger than the second inner diameter, and wherein the first inner diameter tapers to the second inner diameter in a location between a proximal end of the infusion sheath and the distal end of the infusion sheath.

Embodiment 18: The system according to embodiment 17, wherein the second inner diameter forms a fluid tight seal with the outer diameter of the energy probe, such that when a portion of the energy probe is in a first position disposed within the first region of the infusion sheath having the second inner diameter, the fluid does not flow from the inner lumen of the infusion sheath through the distal end of the infusion sheath, and wherein, when the energy probe is in a second position at least partially disposed the second region of the infusion sheath having the first inner diameter and not in the first region of the infusion sheath the infusion sheath having the second inner diameter, the fluid in the inner lumen of the infusion sheath is in fluid communication with the target site.

Embodiment 19: The system according to any one of embodiments 1 to 18, comprising an indicator disposed at least partially on or about the energy probe.

Embodiment 20: The system according to embodiment 19, wherein the indicator is a visual distance and/or rotational indicator located on the system that indicates to a user that a distal end of the infusion sheath has been advanced to a location relative to the probe that co-locates the fluid delivery with the energy delivered or withdrawn by the probe.

Embodiment 21: The system according to any one of embodiments 1 to 20, wherein the indicator is an audio indicator configured to notify a user that the distal end of the infusion sheath has been advanced to a location relative to the probe that co-locates the fluid delivery with the energy delivered by the probe.

Embodiment 22: The system according to any one of embodiments 1 to 21, wherein the indicator marks a target location on a proximal end of the system to slidably position the infusion sheath relative to the energy probe to deliver the fluid in a location that is co-located with the energy delivery or withdrawal location of the probe.

Embodiment 23: The system according to any one of embodiments 1 to 22, wherein the infusion sheath is slidable relative to the longitudinal axis of the energy probe.

Embodiment 24: The system according to any one of embodiments 1 to 23, wherein the energy probe is slidable relative to the longitudinal axis of the infusion sheath.

Embodiment 25: The system according to any one of embodiments 1 to 24, wherein the energy probe is a cryoprobe.

Embodiment 26: The system according to any one of embodiments 1 to 25, wherein the infusion sheath comprises a plurality of fluid pathways disposed within a wall of the infusion sheath, wherein the fluid pathways are configured to release the infusion of the therapeutic agent to flow from a first location of the infusion sheath through a second location of the infusion sheath spaced apart from and located distal to the first location.

Embodiment 27: The system according to any one of embodiments 1 to 26, wherein the second location is flush with the distal end of the infusion sheath.

Embodiment 28: The system according to any one of embodiments 1 to 27, wherein the at least one port is an annular orifice.

Embodiment 29: The system according to any one of embodiments 1 to 28, wherein the infusion sheath comprises a polymer.

Embodiment 30: The system according to any one of embodiments 1 to 29, wherein the infusion sheath comprises a metal.

Embodiment 31: The system according to any one of embodiments 1 to 30, wherein the distance the infusion sheath is slidably moved over the energy probe or the distance the energy probe is moved while at least partially within the infusion sheath is controlled by moving a guide within a slot to a target distance, wherein the guide extends from the energy probe or the infusion sheath and the slot is at least partially disposed in or extending from the corresponding surface of the energy probe or the infusion sheath.

Embodiment 32: The system according to any one of embodiments 1 to 31, wherein the infusion sheath has a first inner diameter located at a proximal end of the infusion sheath and a second inner diameter located at a distal end of the infusion sheath.

Embodiment 33: The system according to embodiment 32, wherein the first inner diameter is larger than the second inner diameter, and wherein the first inner diameter tapers to the second inner diameter in a location between the proximal end of the infusion sheath and the distal end of the infusion sheath.

Embodiment 34: The system according to embodiment 33, wherein the second inner diameter is substantially the same as the outer diameter of the energy probe, such that when a portion of the energy probe is disposed within the second inner diameter, fluid does not flow from the inner lumen of the infusion sheath through the distal end of the infusion sheath, and wherein the first inner diameter is larger than the outer diameter of the energy probe, such that when the energy probe is at least partially disposed only in the portion of the energy probe having the first inner diameter, the inner lumen of the infusion sheath is in fluid communication with the target site.

Embodiment 35: The system according to any one of embodiments 1 to 34, wherein the infusion sheath comprises a plurality of fluid pathways disposed through a wall of the infusion sheath, wherein the fluid pathways are in fluid communication with a source of the fluid at the proximal end of the system, and each pathway is in fluid communication with an associated opening at the distal end of the infusion sheath.

Embodiment 36: The system according to embodiment 35, wherein the opening is selected from a group consisting of a slit and an annular orifice.

Embodiment 37: A method for co-locating energy delivery or withdrawal and fluid delivery to tissue at a target site in a patient, the method comprising: disposing a cryolysis system comprising an energy probe coaxial with and positioned within a lumen of an infusion sheath at least partially into a patient such that a distal end of the energy probe is disposed in the target site; providing energy from the energy probe to the tissue, or vice versa, thereby lysing at least a portion of a plurality of cells in the target site; slidably moving the infusion sheath in a distal direction along a longitudinal axis of the energy probe from a first position to a second position, wherein the second position is spaced apart from the first position; delivering an infusion of a fluid to the target site through the infusion sheath; and removing the infusion sheath and the energy probe from the target site.

Embodiment 38: The method of embodiment 37, comprising delivering the fluid out one or more openings in the distal end of the infusion sheath, wherein such openings are disposed radially through a wall of the infusion sheath to be in fluid communication with a fluid pathway within a lumen of the infusion sheath that extends to a proximal end of the cryolysis system to a fluid source.

Embodiment 39: The method of embodiment 38, wherein the one or more openings is or are slits.

Embodiment 40: The method of any one of embodiments 37 to 39, wherein delivering the fluid and slidably moving the infusion sheath are done concurrently.

Embodiment 41: The method of embodiment 40, comprising slidably moving the infusion sheath in a proximal direction along the longitudinal axis of the energy probe from the second toward the first position, wherein delivering the fluid and slidably moving in this proximal direction are done concurrently.

Embodiment 42: The method of embodiment 41, comprising slidably moving the infusion sheath distal to the second position to a third position, wherein when in such third position buckling of the infusion sheath is reduced relative to when in the first or second positions and/or mechanical strength of the system at the location of the energy probe is enhanced relative to the energy probe alone without the sheath distal advancement, wherein the enhanced mechanical strength is provided about the distal end of the energy probe upon disposing the cryolysis system in the patient.

Embodiment 43: The method of any one of embodiments 37 to 42, wherein the infusion sheath is in the third position when disposed at least partially into the patient.

Embodiment 44: The method of any one of embodiments 37 to 43, wherein a distal end of the infusion sheath is located distal to a distal end of the energy probe when in the third position.

Embodiment 45: The method of any one of embodiments 37 to 44, wherein a distal end of the infusion sheath is located proximal to a distal end of the energy probe when in the third position.

Embodiment 46: The method of any one of embodiments 37 to 45, wherein a distal end of the infusion sheath is located at a location distal to the distal end of the energy probe when in the second position.

Embodiment 47: The method of any one of embodiments 37 to 46, wherein a distal end of the infusion sheath is located at a location proximal to the distal end of the energy probe when in the second position.

Embodiment 48: The method of any one of embodiments 37 to 47, comprising sliding a protrusion extending from the exterior wall of the energy probe within a channel disposed in the infusion sheath to slidably move the infusion sheath from the first position to the second position, wherein the channel is configured to slidably couple with the protrusion, and optionally, wherein the protrusion coupled with the channel is configured to prevent circumferential rotation of the infusion sheath relative to the energy probe.

Embodiment 49: The method of any one of embodiments 37 to 48, comprising sliding a protrusion extending from the exterior surface of the energy probe within a channel disposed at least partially through a wall of the infusion sheath to slidably move the infusion sheath from the first position to the second position, wherein the channel is configured to slidably couple with the protrusion, and optionally, wherein the protrusion coupled with the channel is configured to prevent circumferential rotation of the infusion sheath relative to the energy probe.

Embodiment 50: The method of any one of embodiments 37 to 49, comprising determining the second position by aligning a portion of the proximal end of the sheath relative to a portion of a proximal end of probe according to an audio, tactile, or visual indicator.

Embodiment 51: The method of embodiment 50, wherein the indicator marks a target location to slidably align the infusion sheath and the energy probe thereby co-locating delivery of the fluid with the location of energy delivered or withdrawn by the probe.

Embodiment 52: The method of any one of embodiments 37 to 51, wherein delivering the infusion of a therapeutic agent to the target tissue occurs through a valve configured to connect an infusion tube with a proximal end of the infusion sheath, so as to provide fluid through a pathway within the infusion sheath and out the one or more openings of the infusion sheath.

Embodiment 53: The method of any one of embodiments 37 to 52, wherein the energy probe is a cryoprobe and providing or withdrawing energy to or from the tissue at the tissue site at least partially freezes the tissue thereby lysing at least a portion of the plurality of cells in the target site.

Embodiment 54: The method of any one of embodiments 37 to 53, wherein delivering the infusion of a fluid to the target tissue comprises opening a valve at the proximal end of the system in the fluid pathway of the fluid that connects an infusion fluid source with a proximal end of the infusion sheath.

Embodiment 55: The method of any one of embodiments 37 to 54, wherein the infusion sheath is configured to form a seal with the energy probe when in the first position such that the infusion of therapeutic agent is retained in the infusion sheath, and wherein the infusion sheath is configured to deliver the infusion of therapeutic agent to the target tissue when in the second position.

Embodiment 56: The method of any one of embodiments 37 to 55, wherein a distal end of the infusion sheath is located proximal to the distal end of the energy probe when in the first position.

In further embodiments according to this disclosure, there are provided several embodiments of the system described herein above wherein movement of the infusion sheath relative to the energy probe is achieved by mechanical means to achieve precise location of the distal end of the infusion sheath with the location treated by the tip of the energy probe, to ensure co-location of infusion delivery with the site of cellular disruption. These mechanisms, as described herein below, are referred to as “Sheath Advancement Mechanisms”, or “SAMs”. In preferred embodiments of this aspect of the disclosure, the SAMs not only achieve precise delivery of infusions to the target site treated by an energy probe, but, as taught herein, when unitary with an ergonomic handle to manage the OID with its inputs of cryogen or energy (as in PEF), energy probe, infusion sheath and infusion supply, the added benefit of enhanced comfort, reduced potential for error as all elements required for treatment are in place at the start of a procedure, and increased accuracy may be achieved, whether using an angled cryoprobe, a straight cryoprobe, or the like.

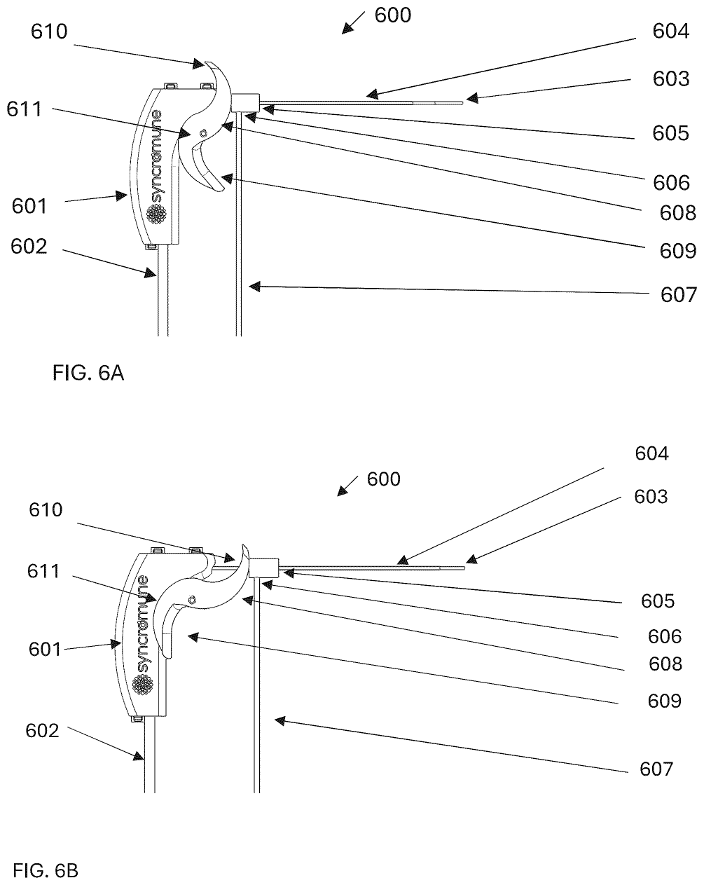

In a first such embodiment, 600, (embodiment 57 herein) shown in FIG. 6A, there may be provided an ergonomic handle 601 (also referred to as “handle 601”). Handle 601 may have a curved outer surface which may be designed to sit in an operator's hand. Handle 601 may have a straight or curved inner surface opposite an outer surface that may allow for enhanced grasping capabilities of handle 601 by an operator. In some embodiments, an outer surface and/or inner surface of handle 601 may be padded. Handle 601 may have a lumen near a bottom portion of handle 601 that may be sized to allow a passage of tube 602 therethrough. Tube 602 may be cylindrical and may have a lumen therethrough of which cryogen may pass. For instance, tube 602 may allow for a passage of cryogen through handle 601 and to energy probe 603. In some embodiments, the energy probe 603 may be selected from the group consisting of argon, nitrous oxide, carbon dioxide, and liquid nitrogen.

Energy probe 603 may be cylindrical, ovular, rectangular, or other shapes. In some embodiments, energy probe 603 may terminate at a rounded tip. A tip or other part of energy probe 603 may be designed to come into contact with a target site and/or target tissue. A tip of energy probe 603 may have a diameter of about, but not limited to, about 1 mm to about 5 mm or greater. Energy probe 603 may be configured to withdraw energy, such as thermal energy, from a target site and/or target tissue through circulation of cryogen through energy probe 603, thereby reducing the temperature of the target site and/or target tissue. For instance, energy probe 603 may reach temperatures of about, but not limited to, −40° C. to about −60° C. or less than about 60° C. Those skilled in the art are aware that cryogen flow rates through tube 602 have to be adapted for each type of cryogen, pressure, and probe to obtain the results as required herein. In some embodiments, such as when argon is used as the cryogen, the pressure from the source may be as high as 4,000 psi, or higher.

Energy probe 603 may be cylindrical, ovular, rectangular, or other shapes. In some embodiments, energy probe 603 may terminate at a rounded tip or a sharp tip to assist in penetrating tissue. A tip or other part of energy probe 603 may be designed to come into contact with a target site and/or target tissue. A tip of energy probe 603 may have a diameter of about, but not limited to, about 1 mm to about 5 mm or greater. Energy probe 603 may be configured to reduce a temperature of a target site and/or target tissue through circulation of cryogen through energy probe 603. For instance, energy probe 603 may reach temperatures of about, but not limited to, −40° C. to about −60° C. or less than about −60° C.

Energy probe 603 may be cylindrical, ovular, rectangular, or other shapes. Energy probe 603 may be disposed within an infusion sheath 604. Infusion sheath 604 may be a cylindrical object that may extend distally away from handle 601. Infusion sheath 604 may provide a fluidic pathway through a lumen therein for fluid to travel distally and exit through openings at the distal portion of infusion sheath 604. Fluid may be, but is not limited to, therapeutic agents such as immunotherapeutic drugs including monoclonal antibodies (mAbs) that block immune checkpoint inhibitor molecules such as PD-1, PD-L1 and CTLA-4, TLR-9 agonists (CpG oligomers), agonistic anti-CD40 mAbs, anti-OX40 antibodies, cancer therapeutic agents, or any single drug or multiple mixture of drugs, without limitation. In some embodiments, infusion sheath 604 may deliver fluid around energy probe 603. For instance, energy probe 603 may come into contact with a target site and/or target tissue and may reduce a temperature of the target site and/or target tissue. Energy probe 603 may be configured to withdraw energy, such as thermal energy, from a target site and/or target tissue through circulation of cryogen through energy probe 603, thereby reducing the temperature of the target site and/or target tissue. Alternatively, energy probe 603 may be configured to emit energy to a target site and/or target tissue, such as radiofrequency energy, microwave energy, ultrasound energy, electrical energy for irreversible electroporation (IRE) or pulsed electric fields (PEF), thereby causing cellular lysis through energy delivery rather than energy withdrawal. Simultaneously or sequentially, fluid may be delivered to the target site and/or target tissue via infusion sheath 604. Operation of energy probe 603 and/or delivery of fluid via infusion sheath 604 may be controlled via a controller that may be part of or sperate from handle 601. A controller may be, but is not limited to, a microprocessor-based control unit, a pneumatic control system, a hydraulic control system, a mechanical control system with manual actuators, an electronic control panel with digital displays and input controls, a programmable logic controller (PLC), a computer-based control interface, or any combination thereof. In some embodiments, the system may include one or more sensors configured to monitor various parameters during operation. Such sensors may include, but are not limited to, temperature sensors disposed on or within the energy probe 603 to monitor the temperature at the target site. In some embodiments, the temperature sensor may be specifically configured to detect temperatures of the target site and/or target tissue to provide real-time thermal monitoring during cryogenic treatment. Additional sensors may include pressure sensors to monitor cryogen pressure within the system, flow sensors to monitor cryogen flow rates through tube 602, fluid level sensors to monitor the volume of therapeutic agents in infusion reservoirs, proximity sensors to detect the position of the infusion sheath 604 relative to the energy probe 603, force sensors to monitor insertion force during placement of the system into target tissue, impedance sensors to monitor tissue characteristics at the target site, thermal sensors to monitor heat transfer rates during cryogenic treatment, vibration sensors to detect mechanical feedback from tissue interaction, and optical sensors to provide visual feedback of treatment progress.

In some embodiments, the controller may be configured to receive sensor data from one or more of these sensors and automatically adjust system operation based on the received sensor data. For example, the controller may automatically modulate cryogen flow rates based on temperature sensor readings to maintain optimal treatment temperatures, adjust infusion rates of therapeutic agents based on pressure sensor feedback, terminate treatment cycles when predetermined temperature thresholds are reached as measured by temperature sensors, modify freeze-thaw cycle timing based on real-time thermal sensor data, alert operators when sensor readings indicate potential system malfunctions or suboptimal treatment conditions, automatically position the infusion sheath 604 based on proximity sensor feedback to ensure co-location with the treatment zone, and provide real-time feedback to operators through visual, audible, or tactile indicators based on sensor data analysis. The controller can operate freeze and thaw cycles to control the timing and duration of cryogenic treatment phases. In some embodiments, handle 601 may include one or more user interface elements that may be coupled to a controller. User interface elements may include, but are not limited to, buttons, knobs, triggers, or other forms of user interface elements. Upon interaction with one or more user interface elements, a controller may operate a fluid pump or other device to cause cryogen to circulate throughout energy probe 603 and/or to cause fluid to pass through infusion sheath 604. In some embodiments, handle 601 may have a dedicated button for cryogen delivery and another dedicated button for therapeutic agent delivery. In some embodiments, operation of delivery of cryogen and/or immunotherapy agents may occur in a predetermined sequence. For instance, an operator may interact with a single user interface element which may cause a controller to pump cryogen throughout an interior of energy probe 603 for an amount of time and then deliver therapeutic agents via infusion sheath 604 for another amount of time.

In some embodiments, a non-limiting example of operation may include the following sequence: an operator may position the cryolysis system 600 at a target site, such as a tumor, and activate the controller to initiate cryogen flow through tube 602 to energy probe 603 for a predetermined duration, such as 2-5 minutes, to achieve cellular lysis within the target tissue. Following completion of the cryolysis cycle and thawing of the target site, the operator may then activate the sheath advancement trigger 608 to advance infusion sheath 604 to the treatment zone, and subsequently activate the controller to deliver therapeutic agents, such as immunotherapeutic drugs, through infusion tube 607 and infusion sheath 604 to the lysed tissue area for a predetermined infusion period, such as 5-15 minutes, thereby co-locating the therapeutic agent delivery with the site of cellular disruption.

Infusion sheath 604 may extend by about, but not limited to, 1 inch to about 12 inches or greater. In some embodiments, the infusion sheath is between about 7 cm to about 24 cm. In some embodiments, infusion sheath 604 may be telescopic. The telescopic design may allow the infusion sheath 604 to be extended or retracted by sliding nested tubular sections relative to one another, thereby providing adjustable length control during the procedure. This telescopic functionality may enable precise positioning of the distal end of the infusion sheath 604 relative to the energy probe 603 without requiring complete replacement of the sheath assembly. For instance, infusion sheath 604 may be able to increase in length with extension of subsequently smaller sub-parts. In this drawing, the coupling of the cryogen carrying tube 602 with the energy probe 603 is not evident from the outside of the handle 601, but a coupling of these elements may be disposed within the handle 601, as shown in further detail in additional embodiments described herein below. Sheath 604 may be retained by ferrule 605. Ferule 605 may be a component that may mechanically couple infusion sheath 604 to a portion of handle 601. Ferrule 605 may include 606. Port 606 may be an opening within ferrule 605 that may allow for insertion of one or more components. For instance, tube 607 may be coupled to Ferule 605 via port 606. Tube 607 may be connected to an infusion reservoir, pump, syringe or the like which may hold the infusate until ready to be delivered through infusion tube 607 and ferrule 605. A sheath advancement trigger 608 may be provided which, when a lower portion of the trigger 609 thereof is squeezed by a user, an upper portion 610 thereof is made to advance ferrule 605, by rotation of the trigger about pin fulcrum point 611. Advancement of the ferrule 605 which may retain the sheath 604 may cause the sheath 604 to be advanced a precise distance over the energy probe 603 to position the terminal end of the sheath 604 at or very near to the terminal end of the energy probe 603, such that when infusion is initiated through infusion tube 607, through ferrule 605 into infusion sheath 604 and out the distal end thereof to thereby co-locate the site of infusion with the site of energy probe treatment within a target zone within a tissue treated by energy probe 603, as shown in FIG. 6B.

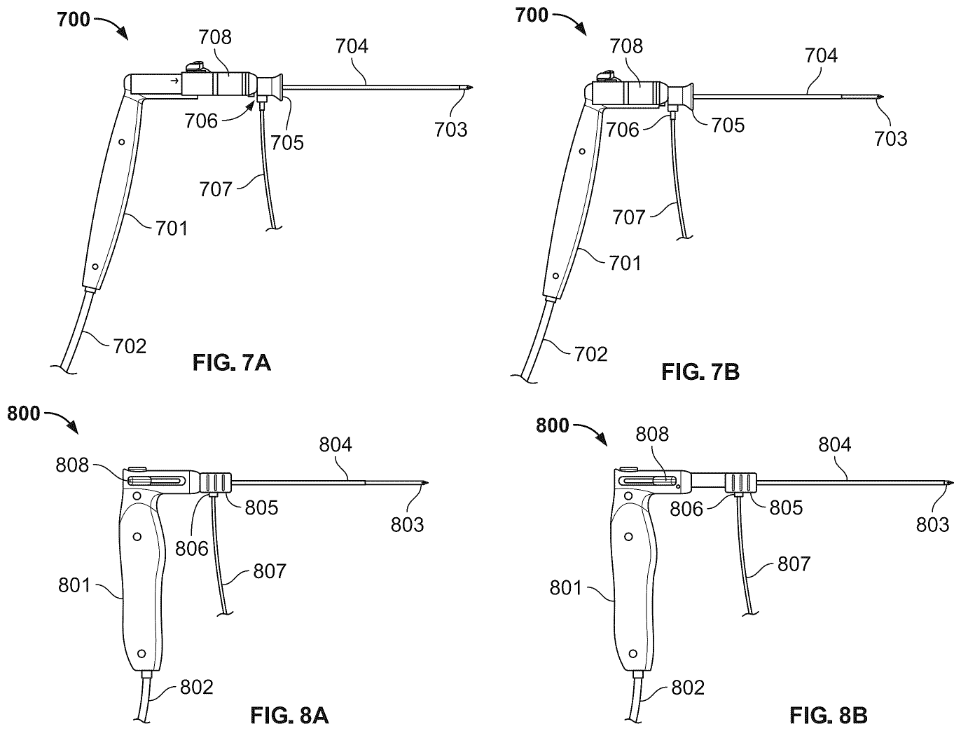

In a second such embodiment 700 (embodiment 58 herein), shown in FIG. 7B, there may be provided an ergonomic handle 701, through which a tube 702 passes for carrying cryogen to an energy probe 703 which itself is disposed within an infusion sheath 704. In this drawing, the coupling of the cryogen carrying tube 702 with the energy probe 703 is not evident from the outside of the handle 701, but a coupling of these elements is disposed within the handle 701. Further, as shown in this embodiment, infusion sheath 704 may be retained by ferrule 705 which may include a port 706 coupled with infusion tube 707 which may be connected to an infusion reservoir, pump, syringe or the like which may hold the infusate until ready to be delivered through infusion tube 707 and ferrule 705. A sheath advancement push tab 708 may be provided on the top of the handle 701 which, in some embodiments, when pushed forward by a user a distance permitted by the push tab, may advance the ferrule 705 which may retain the sheath 704 and may cause the sheath 704 to be advanced a precise distance over the energy probe 703 to position the terminal end of the sheath 704 at or very near to the terminal end of the energy probe 703, such that when infusion is initiated through infusion tube 707, through ferrule 705 into infusion sheath 704 and out the distal end thereof to thereby co-locate the site of infusion with the site of energy probe treatment within a target zone within a tissue treated by energy probe 703, as shown in FIG. 7A. The push tab 708 may be configured with tactile feedback mechanisms to provide the user with confirmation of proper advancement distance. In some embodiments, the push tab 708 may include detents or click stops that correspond to predetermined positions of the infusion sheath 704 relative to the energy probe 703. The push tab 708 may be spring-loaded to return to its original position after advancement, allowing for repeated cycles of sheath advancement and retraction during a procedure. Additionally, the push tab 708 may be designed with ergonomic contours to facilitate comfortable operation by the user during extended procedures. A precise distance may be a distance of about, but not limited to, between about 1 cm to about 5 cm, or between about 2 cm to about 4 cm, or about 3 cm.

In a third such embodiment 800 (embodiment 59 herein), shown in FIG. 8A, there may be provided an ergonomic handle 801, through which a tube 802 passes for carrying cryogen to an energy probe 803 which itself is disposed within an infusion sheath 804. In this drawing, the coupling of the cryogen carrying tube 802 with the energy probe 803 is not evident from the outside of the handle 801, but a coupling of these elements is disposed within the handle 801. Further, as shown in this embodiment, infusion sheath 804 may be retained by ferrule 805 which may include a port 806 coupled with infusion tube 807 which may be connected to an infusion reservoir, pump, syringe or the like which may hold the infusate until ready to be delivered through infusion tube 807 and ferrule 805 into infusion sheath 804. A sheath advancement push tab 808 may be provided on the side of the handle 801 which, in some embodiments, when pushed forward by a user a distance permitted by the push tab, may advance the ferrule 805 which may retain the sheath 804 and may be configured as a sliding element that slides along a track or groove disposed on the side of the handle 801. The sliding motion of the push tab 808 may be bidirectional, such that moving the push tab 808 forward may cause the infusion sheath 804 to extend distally over the energy probe 803, while moving the push tab 808 backward may cause the infusion sheath 804 to retract proximally relative to the energy probe 803. The sliding mechanism may include mechanical linkages or cam systems that translate the linear motion of the push tab 808 into corresponding movement of the ferrule 805 and infusion sheath 804. This bidirectional control allows the user to precisely position the infusion sheath 804 for optimal co-location of the infusate delivery with the energy probe treatment zone. In some embodiments, the push tab 808 may cause the sheath 804 to be advanced a precise distance over the energy probe 803 to position the terminal end of the sheath 804 at or very near to the terminal end of the energy probe 803, such that when infusion is initiated through infusion tube 807, through ferrule 805 into infusion sheath 804 and out the distal end thereof to thereby co-locate the site of infusion with the site of energy probe treatment within a target zone within a tissue treated by energy probe 803, as shown in FIG. 8B.

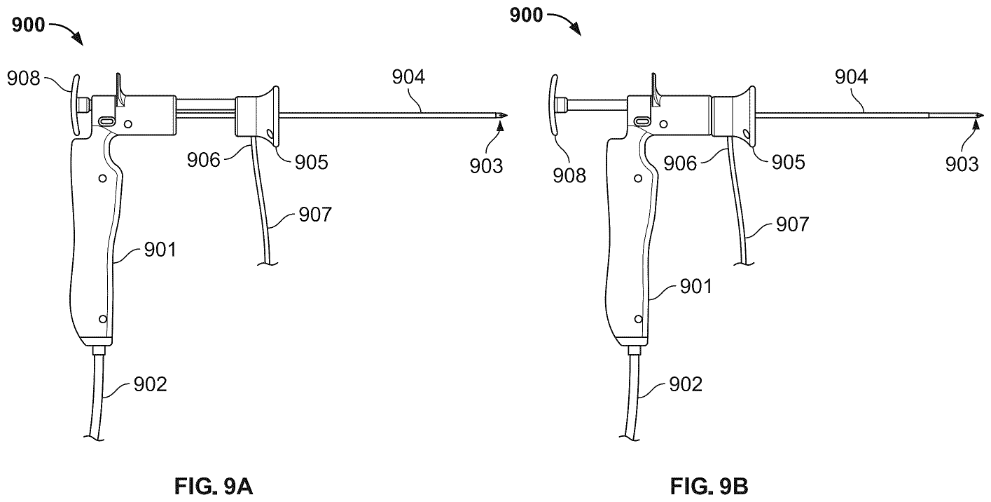

In a fourth such embodiment 900 (embodiment 60 herein), shown in FIG. 9B, there may be provided an ergonomic handle 901, through which a tube 902 passes for carrying cryogen to a cryoprobe 903 which itself is disposed within an infusion sheath 904. In this drawing, the coupling of the cryogen carrying tube 902 with the cryoprobe 903 is not evident from the outside of the handle 901, but a standard coupling of these elements is disposed within the handle 901. Further, as shown in this embodiment, infusion sheath 904 may be retained by ferrule 905 which may include a port 906 coupled with infusion tube 907 which may be connected to an infusion reservoir, pump, syringe or the like which may hold the infusate until ready to be delivered through infusion tube 907 and ferrule 905 into infusion sheath 904. A sheath advancement push plunger 908 may be provided on the rear of the handle 901 which, in some embodiments, when pushed forward by a user a distance permitted by the plunger 908, may advance the ferrule 905 which may retain the sheath 904 may cause the sheath 904 to be advanced a precise distance over the cryoprobe 903 to position the terminal end of the sheath 904 at or very near to the terminal end of the cryoprobe 903, such that when infusion is initiated through infusion tube 907, through ferrule 905 into infusion sheath 904 and out the distal end thereof to thereby co-locate the site of infusion with the site of cryoprobe treatment within a target zone within a tissue treated by cryoprobe 903, as shown in FIG. 9A. The push plunger 908 may be configured for bidirectional movement, such that pushing the plunger 908 distally away from the operator may advance the infusion sheath 904, while retracting the push plunger 908 proximally towards the operator and away from the handle 901 may cause the infusion sheath 904 to be retracted over the cryoprobe 903. At the conclusion of the cycle, the sheath advancement push plunger 908 may be retracted, which may also cause the infusion sheath 904 to be retracted over the cryoprobe 903.

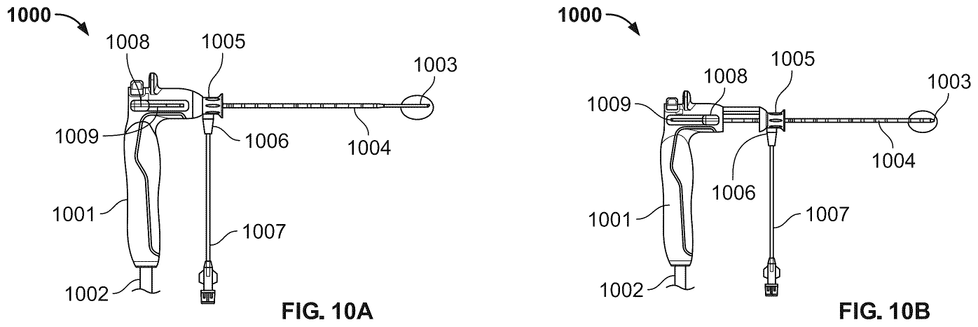

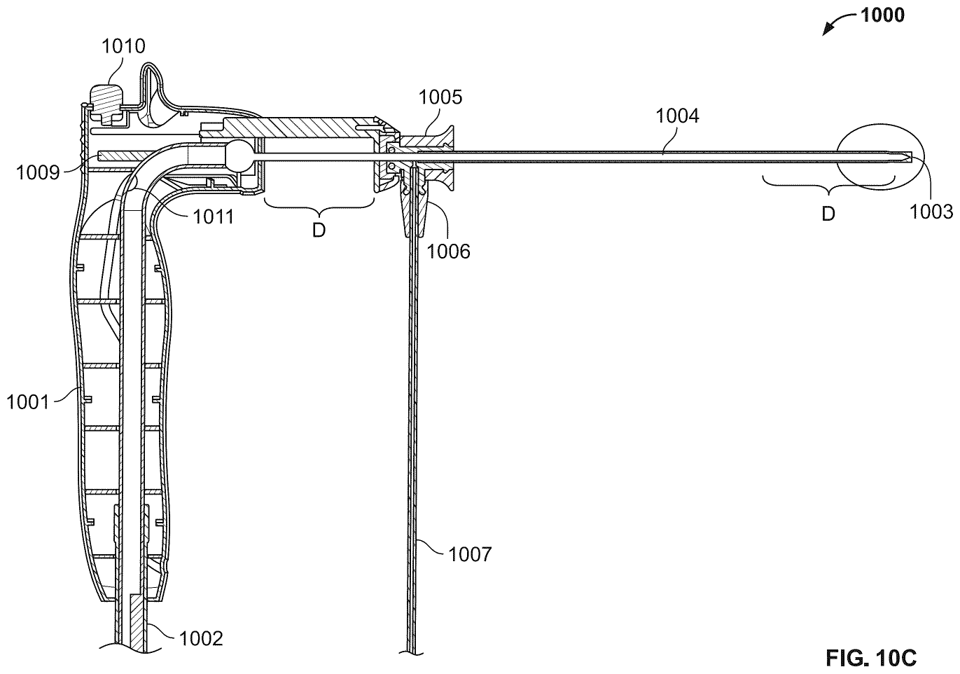

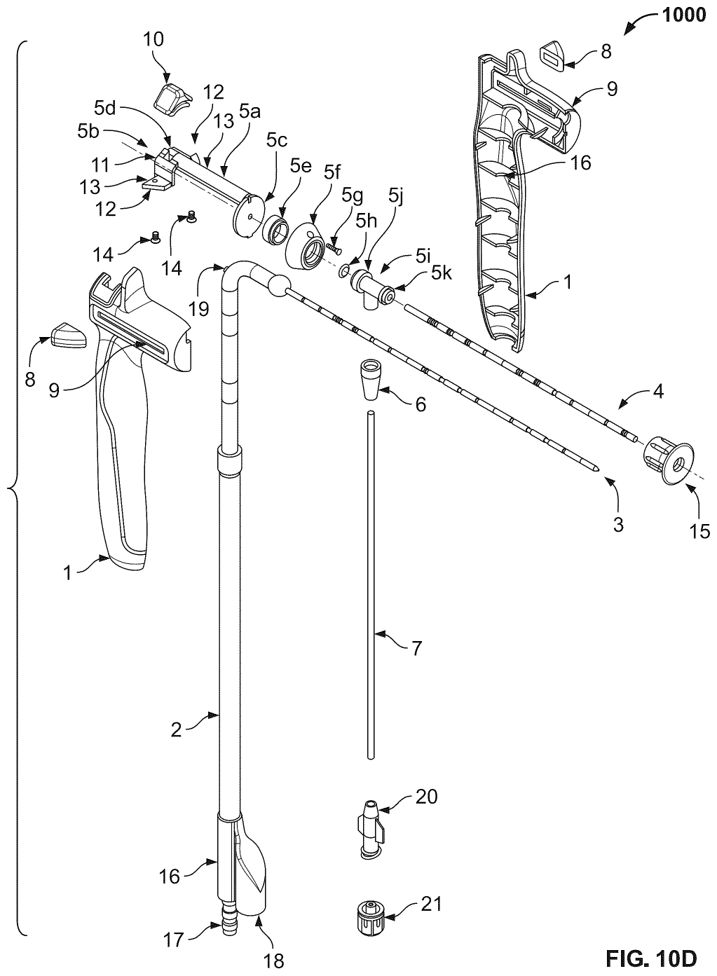

In a fifth such embodiment 1000 (embodiment 61 herein), shown in FIG. 10A, there is provided an ergonomic handle 1001, through which a tube 1002 passes for carrying cryogen to an energy probe 1003 which itself is disposed within an infusion sheath 1004.

In FIG. 10A, the coupling of the cryogen carrying tube 1002 with the cryoprobe 1003 is not evident from the outside of the handle 1001, but a coupling of these elements is disposed within the handle 1001. Further, as shown in this embodiment, infusion sheath 1004 may be retained by ferrule 1005 which may include a port 1006 coupled with infusion tube 1007 which may be connected to a control module, including, generally, a generator, infusion reservoir, pump, syringe or the like which may hold the infusate until ready to be delivered through infusion tube 1007 and ferrule 1005 into infusion sheath 1004. A sheath advancement push tab 1008 may be provided on either or each side of the handle 1001 which, in some embodiments, when pushed forward by a user a distance permitted by the push tab 1008, riding in a guide 1009 through the housing, may advance the ferrule 1005 which may retain the sheath 1004 and may cause the sheath 1004 to be advanced a precise distance over the cryoprobe 1003 to position the terminal end of the sheath 1004 at or very near to the terminal end of the cryoprobe 1003, as in FIG. 10B and FIG. 10C, such that when infusion is initiated through infusion tube 1007, through ferrule 1005 into infusion sheath 1004 and out the distal end thereof to thereby co-locate the site of infusion with the site of cryoprobe treatment within a target zone within a tissue treated by cryoprobe 1003, as shown in FIG. 10B and FIG. 10C. At the conclusion of the cycle, as shown in FIG. 10A, the sheath advancement push tab 1008 may be retracted, which may also cause the infusion sheath 1004 to be retracted over the cryoprobe 1003, after which the entire apparatus may be withdrawn as a single unit.

As shown in FIG. 10C, there may be provided a sectional view of the embodiment 1000, showing the interior of handle 1001, through which a tube 1002 passes for carrying cryogen to an energy probe 1003 which itself is disposed within an infusion sheath 1004. In some embodiments, tube 1002 may include a temperature measuring thermocouple, or the like, with associated wiring, which likewise may be bound to 1002. In this drawing, the coupling of the cryogen carrying tube 1002 with the energy probe 1003 may be disposed within the handle 1001. Further, as shown in this embodiment, infusion sheath 1004 may be retained by ferrule 1005 which may include a port 1006 coupled with infusion tube 1007 which may be connected to an infusion reservoir, pump, syringe or the like which may hold the infusate until ready to be delivered through infusion tube 1007 and ferrule 1005 into infusion sheath 1004. A sheath advancement push tab 1008 may not be evident in this depiction, but may be provided on each side of the handle 1001 which, in some embodiments, when pushed forward by a user a distance permitted by the push tab 1008, riding in a guide 1009 through the housing, may advance the ferrule 1005 a distance “D” which may retain the sheath 1004 and may cause the sheath 1004 to be advanced a precise distance “D” over the energy probe 1003 to position the terminal end of the sheath 1004 at or very near to the terminal end of the energy probe 1003, as in FIG. 10B and FIG. 10C, such that when infusion is initiated through infusion tube 1007, through ferrule 1005 into infusion sheath 1004 and out the distal end thereof to thereby co-locate the site of infusion with the site of energy probe treatment within a target zone within a tissue treated by energy probe 1003, as shown in FIGS. 10B and 10C. At the conclusion of the cycle, as shown in FIG. 10A, the sheath advancement push tab 1008 may be retracted, which may also cause the infusion sheath 1004 to be retracted over the energy probe 1003.

FIG. 10D may provide an exploded view of embodiment 1000, with all elements shown with numbering which should be added to 1000, thus, element 1 is actually element 1001. Additional detail may be provided to ensure that those skilled in the art are able to assemble this and related embodiments to operate according to this disclosure and the attached claims. In some embodiments, handle, left and right housing components 1, 2, energy probe sub-assembly including a coupling 16, comprising both a high-pressure gas or liquid tight coupling 17 and an electrical coupling 18, preferably in a unitary configuration for simultaneous coupling and decoupling of these key components for energy probe operation to and from a control unit's matching couplings. Thermocouple 19 may be shown, which may be connected via appropriate wiring running to electrical coupling 18. Coupling 16 may be coupled with a control panel which may control the provision and receipt of electrical signals to and from 16, and cryogen to and from 17; 3 energy probe; 4 sheath; 5 sheath advancement mechanism, comprising, in this preferred embodiment, in addition to push tab 8, push tab guide or channel 9: pusher rod 5a, which comprises a proximal end 5b, a flat distal end 5c, wherein pusher rod 5a connects the proximal 5b and distal 5c ends. A mount retention groove 5d may be provided at or near the proximal end 5b, which may mate with pusher rod mount 11, which may have two wings 12 on either side, with screw holes 13 in each wing. Each wing 13 may fit through guide grooves 9 in either side of handle 1, and once through guide grooves 9, each push tab 8 may be affixed to each wing 12 via tab to mount fixation screws 14. In this way, pusher rod 5a may be advanced when either push tab 8 on either side of handle 1 is unlocked by releasing lock 10 and pushed toward the distal end of the energy probe 3 as permitted by groove 9. Sub-assembly 5a-5k, when assembled, may form a fluid-tight chamber comprised of: hemostat valve cap 5e; distal flat end 5c of pusher rod 5a; pusher collar 5f; pusher collar to pusher screw 5g; O-ring EPDM 5h; and hemostasis valve 5i. Hemostasis valve 5i may connect to pusher collar 5f, such that when pusher collar to pusher screw 5g is screwed into the distal end 5c of pusher rod 5a, a fluid-tight seal with O-ring EPDM 5h may be formed. When infusion is initiated via tube 7, through ferrule 6, into hemostasis valve 5i, the fluid may exit into sheath 4, affixed at the distal end 5k of hemostasis valve 5i. Boot cover 15 may tighten and affix sheath 4 to the distal end 5k of hemostasis valve 5i. Luer lock port 20 and port cap 21 may be fixed via a mating luer lock to a source of infusion, after the port cap 21 is removed. When fully assembled, handle halves 1 may be brought together with ribs 16 provided to hold energy probe 3 firmly in place. The handle halves 1 may be affixed to each other by glue, welding, screws, or any other convenient method.

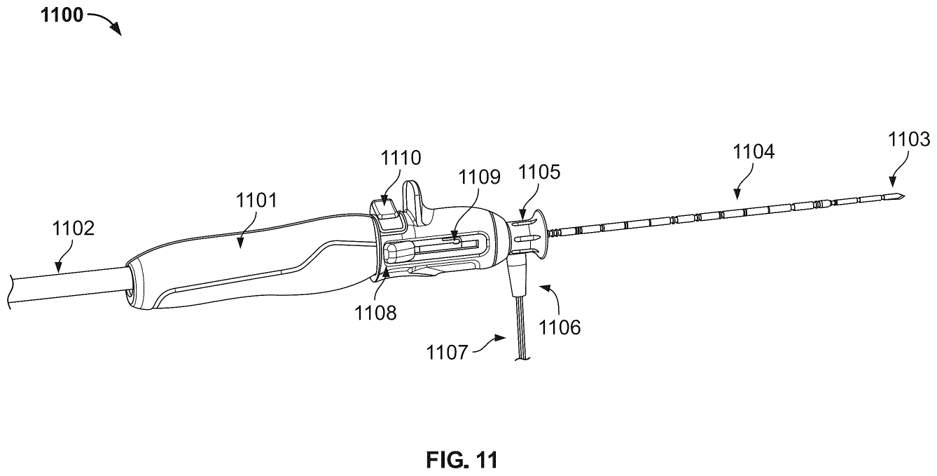

As shown in FIG. 11, there may be provided an analogous unitary energy probe 1103, sheath 1104, handle 1101, sheath advancement mechanism, (comprising at least one or all of elements 1105, 1106, 1107, 1108, 1109, 1110) in combination for use with a cryogen source 1102 connected to a straight, non-angled energy probe 1103, housed within sheath 1104, as described above for the angled probe. In this embodiment push tab 1108 may be configured as a bilateral control mechanism disposed on opposing sides of handle 1101, allowing for ambidextrous operation by the user. Push tab 1108 may be mechanically coupled to the sheath advancement mechanism through a linkage system that translates lateral pressure applied to push tab 1108 into longitudinal movement of infusion sheath 1104 relative to energy probe 1103. The push tab 1108 may include tactile feedback elements such as ridged surfaces or textured grips to provide enhanced user control during operation, particularly when the operator wearing surgical gloves. Push tab 1108 may be spring-loaded to return to a neutral position when not actively engaged, thereby preventing inadvertent advancement of the infusion sheath 1104. Guide slot 1109 may be configured as a precision-machined channel or groove disposed within the housing of handle 1101, designed to constrain and direct the movement of push tab 1108 along a predetermined path. Guide slot 1109 may extend longitudinally along the handle 1101 and may include detent positions or click stops at predetermined intervals corresponding to specific advancement distances of infusion sheath 1104. The guide slot 1109 may be configured with smooth internal surfaces to minimize friction during operation while maintaining precise control over the range of motion of push tab 1108. In some embodiments, guide slot 1109 may include visual indicators or markings that correspond to specific positions of infusion sheath 1104 relative to energy probe 1103, allowing the operator to visually confirm proper positioning during the procedure. The guide slot 1109 may be designed with mechanical stops at both proximal and distal ends to prevent over-travel of push tab 1108 beyond safe operational limits. Lock tab 1110 may be provided as a safety mechanism to secure push tab 1108 in a desired position and prevent unintended advancement of sheath 1104 during critical phases of the procedure. Lock tab 1110 may be configured as a pivoting or sliding element that engages with push tab 1108 or guide slot 1109 to mechanically constrain movement of the sheath advancement mechanism. In some embodiments, lock tab 1110 may include a spring-loaded mechanism that automatically engages when push tab 1108 reaches predetermined positions, requiring deliberate user action to disengage the lock. Lock tab 1110 may be color-coded or include visual indicators to clearly communicate its engagement status to the operator. The lock tab 1110 may be positioned for easy access by the operator's thumb or index finger while maintaining a secure grip on handle 1101. In operation, the user may first position the system with energy probe 1103 at the target site, engage lock tab 1110 to secure the initial position, perform the energy delivery or withdrawal procedure, then disengage lock tab 1110 and actuate push tab 1108 to advance infusion sheath 1104 along guide slot 1109 to the treatment zone for precise co-location of fluid delivery with the site of cellular disruption. The coordinated operation of push tab 1108, guide slot 1109, and lock tab 1110 provides a comprehensive control system that ensures accurate, repeatable positioning of infusion sheath 1104 while maintaining safety and case of use throughout the procedure.

In light of the present disclosure, those skilled in the art will appreciate that disclosed and claimed herein is an integrated hand-held device comprising (a) a sheath through which a liquid composition is delivered, where the sheath is coaxial with (b) an energy delivery or energy withdrawal probe for limited lysis of cells constituting a tumor, that is oncolysis, and (c) an under a user's control sheath advancement or retraction structure to advance or retract the sheath a precise distance over the probe, to achieve delivery of said liquid composition to the site of oncolysis when the user advances the sheath said precise distance over the probe. The claimed oncolytic infusion device is used to both achieve limited lysis of cells constituting a tumor and to deliver a liquid composition, comprising a combination of immunomodulatory agents, chemotherapeutic agents, or the like, precisely at the site of oncolysis.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A system for collocating an energy for cell lysis and an infusion of a fluid in a target site including tissue of a subject, the system comprising:
(a) an energy probe configured to emit or withdraw an energy into or from the tissue of the target site thereby causing cell lysis of said tissue; and
(b) an infusion sheath having a lumen configured to at least partially surround the energy probe, wherein the infusion sheath is configured to provide a pathway for the fluid to travel distally within the lumen and exit a distal portion of the infusion sheath,
wherein the probe is coaxial with the infusion sheath and shares a longitudinal axis thereby allowing co-location of the energy from the probe and the infusion of the fluid from the infusion sheath in the target site.

2. The system according to claim 1, wherein the infusion sheath comprises at least one port in the distal portion of the infusion sheath that is in fluid communication with the pathway of the infusion sheath.

3. The system according to claim 2, wherein the at least one port is a slit.

4. The system according to claim 1, wherein the energy probe and the infusion sheath are translatable relative to one another along the longitudinal axis.

5. The system according to claim 1, wherein the pathway and port are configured to deliver the fluid while the probe is within the pathway at the distal end of the infusion sheath.

6. The system according to claim 1, comprising a valve configured to eliminate proximal flow of the fluid during and/or following delivery thereof to the target site.

7. The system according to claim 1, comprising a tube in fluid communication with the pathway of the infusion sheath at a distal end of the tube and in fluid communication with a source of the fluid at a proximal end of the tube.

8. The system according to claim 1, wherein the fluid is a therapeutic agent effective in treating the tissue at the tissue site.

9. The system according to claim 8, wherein the therapeutic agent is a cancer therapeutic agent.

10. The system according to claim 1, wherein the energy induces cryolysis of tissue at the target site.

11. The system according to claim 1 comprising an integrated hand-held device comprising (a) a sheath through which a liquid composition is delivered, where the sheath is coaxial with (b) an energy delivery or energy withdrawal probe for limited lysis of cells constituting a tumor, that is oncolysis, and (c) an under a user's control sheath advancement or retraction structure to advance or retract the sheath a precise distance over the probe, to achieve delivery of said liquid composition to the site of oncolysis when the user advances the sheath said precise distance over the probe.

12. A method for co-locating energy delivery or withdrawal and fluid delivery to tissue at a target site in a patient, the method comprising:
disposing a cryolysis system comprising an energy probe coaxial with and positioned within a lumen of an infusion sheath at least partially into a patient such that a distal end of the energy probe is disposed in the target site;
providing or withdrawing energy from the energy probe to or from the tissue thereby lysing at least a portion of a plurality of cells in the target site;
slidably moving the infusion sheath in a distal direction along a longitudinal axis of the energy probe from a first position to a second position, wherein the second position is spaced apart from the first position;
delivering an infusion of a fluid to the target site through the infusion sheath; and
removing the infusion sheath and the energy probe from the target site.

13. The method of claim 12, comprising delivering the fluid out one or more openings in the distal end of the infusion sheath, wherein such openings are disposed radially through a wall of the infusion sheath to be in fluid communication with a fluid pathway within a lumen of the infusion sheath that extends to a proximal end of the cryolysis system to a fluid source.

14. The method of claim 13, wherein the one or more openings is a slit.

15. The method of claim 12, wherein delivering the fluid and slidably moving the infusion sheath are done concurrently.

16. The method of claim 15, comprising slidably moving the infusion sheath in a proximal direction along the longitudinal axis of the energy probe from the second toward the first position, wherein delivering the fluid and slidably moving in this proximal direction are done concurrently.

17. The method of claim 16, comprising slidably moving the infusion sheath distal to the second position to a third position, wherein when in such third position buckling of the infusion sheath is reduced relative to when in the first or second positions and/or mechanical strength of the system at the location of the energy probe is enhanced relative to the energy probe alone without the sheath distal advancement, wherein the enhanced mechanical strength is provided about the distal end of the energy probe upon disposing the cryolysis system in the patient.

18. The method of claim 17, wherein the infusion sheath is in the third position when disposed at least partially into the patient.

19. The method of claim 17, wherein a distal end of the infusion sheath is located distal to a distal end of the energy probe when in the third position.

20. The method of claim 17, wherein a distal end of the infusion sheath is located proximal to a distal end of the energy probe when in the third position.

21. The method of claim 12, wherein a distal end of the infusion sheath is located at a location distal to the distal end of the energy probe when in the second position.

22. The system according to claim 1, further comprising a sheath advancement mechanism.

23. The system according to claim 22, wherein said sheath advancement mechanism is incorporated into an ergonomic handle.

24. The system according to claim 23 wherein said sheath advancement mechanism comprises (a) a trigger which when squeezed advances said sheath, (b) a plunger which when depressed advances said sheath, or (c) a push tab which when pushed advances said sheath.

25. The system according to claim 1 comprising:
(a) a handle adapted to be handheld or fixed to a mechanical fixture;
(b) a cryoprobe sheath in movable concentric axial alignment with and covering at least a portion of a cryoprobe when inserted into an end of said sheath proximal said handle, such that advancement or retraction of said sheath in relation to a distal end of said cryoprobe causes said distal end of said cryoprobe to emerge from or be covered by a distal end of said sheath;
(c) a sheath advancement and retraction mechanism which permits precise mechanical advancement, retraction, or both, of said sheath with respect to a distal end of said cryoprobe;
(d) a conduit from a source of fluid in sealed fluid communication with said sheath such that when a distal end of said sheath is not advanced beyond a set point in relation to said cryoprobe, fluid from said fluid source is restricted from flowing through said sheath, and when said distal end of said sheath is advanced to a set point in relation to said cryoprobe, fluid from said fluid source is permitted to flow from said fluid source, through and out of said distal end of said sheath; and
(e) a source of compressed cryogenic gas which endothermically expands within said cryoprobe to cause fracture of tissue placed in contact with said cryprobe.

26. The method according to claim 12, further comprising providing a sheath advancement mechanism which is induced to advance said sheath over said energy probe a defined distance.

27. The method according to claim 26, wherein said sheath advancement mechanism is incorporated into an ergonomic handle.

28. The method according to claim 27 wherein said sheath advancement mechanism comprises (a) a trigger which when squeezed advances said sheath, (b) a plunger which when depressed advances said sheath, or (c) a push tab which when pushed advances said sheath.

29. The method according to claim 12 comprising:
(a) a handle adapted to be handheld or fixed to a mechanical fixture;
(b) a cryoprobe sheath in movable concentric axial alignment with and covering at least a portion of a cryoprobe when inserted into an end of said sheath proximal said handle, such that advancement or retraction of said sheath in relation to a distal end of said cryoprobe causes said distal end of said cryoprobe to emerge from or be covered by a distal end of said sheath;
(c) a sheath advancement and retraction mechanism which permits precise mechanical advancement, retraction, or both, of said sheath with respect to a distal end of said cryoprobe;
(d) a conduit from a source of fluid in sealed fluid communication with said sheath such that when a distal end of said sheath is not advanced beyond a set point in relation to said cryoprobe, fluid from said fluid source is restricted from flowing through said sheath, and when said distal end of said sheath is advanced to a set point in relation to said cryoprobe, fluid from said fluid source is permitted to flow from said fluid source, through and out of said distal end of said sheath; and
(e) a source of compressed cryogenic gas or fluid which endothermically expands within said cryoprobe to cause fracture of tissue placed in contact with said cryprobe.
